SEQUENCE LISTING (34 sequences)

SEQ ID NO: 1 - RNA, 27 nt, artificial sequence
  III: hammerhead ribozyme denoted III, upper strand in figure
cgaaacggugaaagccguagguugccc

SEQ ID NO: 2 - RNA, 17 nt, artificial sequence
  hammerhead ribozyme denoted I, lower strand in figure
gggcgacccugaugaga

SEQ ID NO: 3 - RNA, 24 nt, artificial sequence
  FMN aptamer boxed in figure
aggauaugcuucuucggcagaagg

SEQ ID NO: 4 - RNA, 22 nt, artificial sequence
  I-1 class I induction module
gccuuagccuucgggcgacguc

SEQ ID NO: 5 - RNA, 21 nt, artificial sequence
  I-2 class I induction module
gccuugccuucgggcgacguc

SEQ ID NO: 6 - RNA, 21 nt, artificial sequence
  I-3 class I induction module
gcguugccuucgggcgacgcc

SEQ ID NO: 7 - RNA, 18 nt, artificial sequence
  class II induction module
gauggccuucgggcucuc

SEQ ID NO: 8 - RNA, 25 nt, artificial sequence
  theophilline aptamer boxed in figure
auaccagccgaaaggcccuuggcag

SEQ ID NO: 9 - RNA, 24 nt, artificial sequence
  clone cGMP-1
cagcagucguggaaaaacguagcg

SEQ ID NO: 10 - RNA, 25 nt, artificial sequence
  clone cGMP-2
gagaagcuggaaaaacgcaaacacg

SEQ ID NO: 11 - RNA, 23 nt, artificial sequence
  clone cGMP-3
cgcaccaacguucgucggcugca

SEQ ID NO: 12 - RNA, 23 nt, artificial sequence
  clone cGMP-4
accccagaggucagcugcauaac

SEQ ID NO: 13 - RNA, 24 nt, artificial sequence
  clone cGMP-5
gcaccgacgguagcgaggcgauua

SEQ ID NO: 14 - RNA, 22 nt, artificial sequence
  clone cGMP-6
uugcgcgacuacaacgcaauua

SEQ ID NO: 15 - RNA, 21 nt, artificial sequence
  clone cGMP-7
caaugucacucagcacgauua

SEQ ID NO: 16 - RNA, 22 nt, artificial sequence
  clone cGMP-8
cggggcucauagcuugccacgc

SEQ ID NO: 17 - RNA, 25 nt, artificial sequence
  clone cCMP-1
cacagaaaguggugugaaccgggau

SEQ ID NO: 18 - RNA, 25 nt, artificial sequence
  clone cCMP-2
ggauaaggugucugcacuaguggau

SEQ ID NO: 19 - RNA, 24 nt, artificial sequence
  clone cCMP-3
caaaaacggcgacuacccgcauua

SEQ ID NO: 20 - RNA, 24 nt, artificial sequence
  clone cCMP-4
gaguugcgcgcagaaccgccauua

SEQ ID NO: 21 - RNA, 24 nt, artificial sequence
  clone cCMP-5
uagccaacgucagugugcgcauua

SEQ ID NO: 22 - RNA, 25 nt, artificial sequence
  clone cCMP-6
aaaguugcggacuacaacgcaauua

SEQ ID NO: 23 - RNA, 24 nt, artificial sequence
  clone cCMP-7
ugcggacuugcaaugcgccgauua

SEQ ID NO: 24 - RNA, 24 nt, artificial sequence
  clone cAMP-1
ucaguacacggugcagacaaaggu

SEQ ID NO: 25 - RNA, 24 nt, artificial sequence
  clone cAMP-2
ucgaggaggcaggugcaugugggc

SEQ ID NO: 26 - RNA, 23 nt, artificial sequence
  clone cAMP-3
ccccggcgcauuggacgacgagu

SEQ ID NO: 27 - RNA, 23 nt, artificial sequence
  clone cAMP-4
cgaagcugaccaugcucagcggg

SEQ ID NO: 28 - RNA, 24 nt, artificial sequence
  clone cAMP-5
ucgagucuucagaugcauguggga

SEQ ID NO: 29 - RNA, 24 nt, artificial sequence
  clone cAMP-6
gugaguauucaacgugauguggaa

SEQ ID NO: 30 - RNA, 23 nt, artificial sequence
  clone cAMP-7
ucgagaaucaggugcauguggua

SEQ ID NO: 31 - RNA, 22 nt, artificial sequence
  clone cAMP-8
cgacuccgaccaacgggggacg

SEQ ID NO: 32 - DNA, 33 bp, artificial sequence
  primer used in constructs
taatacgactcactatagggcgaccctgatgag

SEQ ID NO: 33 - DNA, 27 bp, artificial sequence
  primer used in constructs
gggcaacctacggctttcaccgtttcg

SEQ ID NO: 34 - DNA, 19 bp, artificial sequence
  primer used in constructs
aaggctcatcagggtcgcc

(19) United States
(12) Patent Application Publication
(10) Pub. No.: US 2006/0121510 A1
(43) Pub. Date: Jun. 8, 2006

(54) Multidomain polynucleotide molecular sensors

(76) Inventors: Ronald R. Breaker, Guilford, CT (US); Garrett A. Soukup, Papillion, NE (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/288,869

(22) Filed: Nov. 28, 2005

Related U.S. Application Data
(63) Continuation of application No. 09/830,905, filed on Aug. 8, 2001, now abandoned.
(60) Provisional application No. 60/106,829, filed on Nov. 3, 1998.
(60) Provisional application No. 60/126,693, filed on Mar. 29, 1999.

(51) Int. Cl. C12Q 1/68; C07H 21/02

(52) U.S. Cl. 435/6; 536/23.1

(57) ABSTRACT

Multidomain polynucleotides responsive to signalling agents are designed and constructed to have at least three domains which can be partially or completely overlapping or nonoverlapping: an actuator (catalytic or reporter) domain, a bridging domain, and a receptor domain. In a typical embodiment, a signalling agent such as a chemical ligand interacts with the receptor domain, which changes conformation or otherwise influences the bridging domain so that the activity, catalytic, or reporter function of the actuator domain is stimulated or inhibited. In some ribozyme embodiments, for example, ligand-specific molecular sensors composed of RNA are created by coupling pre-existing catalytic and receptor domains via novel structural bridges which function such that binding of a ligand to the receptor domain triggers a conformational change within the bridge, and this structural reorganization dictates the activity of the adjoining ribozyme. Processes for allosterically selecting other multidomain polynucleotides typically involve mixing and matching domains to optimize binding or other signal response and/or reporter activity.

### CROSS-REFERENCES TO RELATED APPLICATIONS

This application claims priority benefit of U.S. Application Ser. No. 60/106,829, filed Nov. 3, 1998, and U.S. Application Ser. No. 60/126,683, filed Mar. 29, 1999.

### STATEMENT REGARDING FEDERALLY SPONSORED RESEARCH

This invention was made with partial government support under grants from the NIH (GM57500 and GM59343) and the Defense Advance Research Projects Agency (DARPA). The government has certain rights in the invention.

### BACKGROUND OF THE INVENTION

1. Field of the Invention

This invention relates to a special class of allosteric polynucleotides and processes for generating highly specific polynucleotide sensors with relative ease and efficiency.

2. Description of the Related Art

Mastery of the molecular forces that dictate biopolymer folding and function would allow molecular engineers to participate in the design of enzymes—a task that to date has been managed largely by the random processes of evolution. The reward for acquiring this capability is substantial considering that many applications in medicine, industry and biotechnology demand high-speed enzymes with precisely tailored catalytic functions. ‘Modular rational design’ has proven to be an effective means for conferring additional chemical and kinetic complexity upon existing protein (e.g. 1-4) and RNA enzymes (5-9). This engineering strategy takes advantage of the modular nature of many protein (10) and RNA subdomains (11-13), which can be judiciously integrated to form new multifunctional constructs. The recent discoveries of new catalytic RNA motifs (14, 15) and new ligand-binding motifs (16, 17) have considerably expanded the opportunities for ribozyme engineering.

Modular rational design has been used to create several artificial ribozymes that are activated or deactivated by the binding of specific small organic molecules such as ATP (5,8) and flavin mononucleotide (FMN) (9). Each of these allosteric ribozymes is composed of two independent structural domains: one an RNA-cleaving ribozyme and the other a receptor (or “aptamer”) for a specific ligand. The conformational changes that occur within an aptamer domain upon introduction of the ligand, termed “adaptive binding” (22-25), can trigger kinetic modulation of the adjoining catalytic domain by several different mechanisms that ultimately influence ribozyme folding (7,8).

Several groups of investigators have suggested that ribozymes or other nucleic acids might be used in assays and the like. For example, diagnostics using ribozymes that catalyze the cleavage and release of a non-complementary, labelled nucleic acid co-target marker in the presence of a specific nucleic acid target molecule has been disclosed (43). Nucleic acid molecules which have no catalytic activity without a specific protein or nucleic acid co-factor and feature catalytic activity only in the presence of the same macromolecular co-factor have been disclosed as useful primarily in therapeutics (44). Bioreactive allosteric polynucleotides that modify a function or configuration of the polynucleotide with a chemical effector and/or physical signal were disclosed for biosensors and/or enzymes for diagnostic and catalytic purposes (45).

In nearly all examples reported to date, allosteric ribozymes have been created by joining preexisting ligand-binding domains (or “aptamers”) with ribozyme domains to produce the ligand-responsive construct of choice (9, 65). Since these methods require the use of preexisting ribozyme and ligand-binding structures, the limited number of RNA domains that are currently available restricts the versatility of allosteric ribozyme engineering. Moreover, while modular rational design alone or combined with in vitro selection techniques has been successful in producing allosteric catalysts from pre-existing aptamer and ribozyme motifs, the process can be slow and tedious. Many previously described procedures necessary to identify nucleic acids having specified binding or catalytic properties involve step-wise iterations of binding, partitioning and amplification (46-53). Furthermore, exclusive use of modular rational design precludes the development of allosteric ribozymes controlled by effectors for which no aptamer motifs exist.

### BRIEF SUMMARY OF THE INVENTION

It is an objective of the invention to use the combined application of modular rational design, in vitro selection, and allosteric selection to provide an effective strategy for the rapid generation of precision polynucleotide molecular sensors.

It is another objective of the invention to provide specific ways of employing polynucleotides as novel sensors and as in vivo genetic control elements for the regulation and/or report of gene expression.

It is a further objective of the invention to provide polynucleotide sensing elements for use in a variety of clinical, industrial, agricultural, and environmental analyses.

These and other objectives are accomplished by the present invention, which provides purified functional polynucleotides comprising an actuator domain, a receptor domain, and a bridging domain, wherein a signalling event such as binding of a ligand to the receptor domain triggers a conformational change in the bridging domain which then modulates the catalytic and/or reporter activity of the actuator domain. The domains may be partially or completely overlapping or non-overlapping such that one or more domain functions may be encoded in part by the same polynucleotide sequence. The polynucleotides can comprise RNA and/or RNA analogues or DNA and/or DNA analogues; tripartite ribozymes are illustrated in the examples.

Also provided are processes for screening for multidomain polynucleotide sensors using allosteric selection. In a typical process, a structural component of a multidomain allosteric polynucleotide is replaced with a random-sequence domain to develop new receptor domains or even new actuator domains using in vitro selection. Briefly, using an example process, randomization of the ligand-binding region of a polynucleotide generates new, structurally diverse polynucleotides that can then be screened to interact with other ligands.

Polynucleotide sensors of the invention are employed to qualitatively or quantitatively measure a variety of ligands, including, but not limited to, organic and/or inorganic compounds, metal ions, pharmaceuticals, microbial or cellular metabolites, blood or urine components, components of other bodily fluids, and macromolecules. The sensors can also be employed to respond to electromagnetic signals and/or physical signals such as temperature, light, sound, shock, pH, and ionic conditions. The sensors are attached to a solid support in some embodiments. Also provided are biosensors having multidomain polynucleotides of the invention as sensing elements.

Polynucleotide sensors of the invention may also be used in vivo as genetic control elements that regulate or report gene expression in response to a ligand or signal, including non-invasive diagnostics and gene therapy strategies.

In this aspect, methods of the invention encompass methods for regulating expression of a gene in a cell by operably linking polynucleotides of the invention to genetic molecules of a cell such that the biological or phenotypic activity encoded by the gene is modulated in accordance with modulation of the activity of the actuator domain. In embodiments involving expression of genes using RNA, multidomain polynucleotide sensors may be incorporated in the coding region of mRNA or in close proximity, but also in the 5′-leader or 3′-tail regions. In DNA embodiments, polynucleotide sensors may be incorporated in regions that signal gene destruction as well as gene expression.

Processes for generating ligand-responsive and other multidomain sensors of the invention are also provided by the generation of novel allosteric molecules using modular rational design strategies. In typical embodiments, a necessary structural component of an allosteric ribozyme is replaced with a random-sequence domain to produce polynucleotides having new effector-binding sites or new effector-modulated catalytic domains that can be screened using in vitro selection. Briefly, in one embodiment, for example, randomization of the ligand-binding region of an allosteric ribozyme generates new structural diversity and a family of structurally parallel polynucleotides that are screened for their efficiency in responding to, and/or reporting, ligand binding. By using this allosteric selection strategy, new allosteric ribozymes with specificity for a great variety of effector molecules are generated.

Methods for using multidomain polynucleotide sensors of the invention are correspondingly provided, as are processes for preparing polynucleotides that are responsive to the presence or absence of a signalling agent such as a chemical ligand that binds to the receptor domain. Also provided are analytical sensors having multidomain polynucleotides of the invention as sensing elements.

### BRIEF DESCRIPTION OF THE FIGURES

FIG. 1 shows the design of initial populations for allosteric selection of aptamer domains and allosteric hammerhead ribozymes (SEQ ID NOs 1 and 2). A random-sequence region that is x nucleotides (where x=any length) is appended to the catalytic nucleic acid motif directly (A) or through an existing communication module such as the class I induction module (B). N represents any nucleotide identity and the arrowhead indicates the site of cleavage within the hammerhead ribozyme domain. In alternate embodiments (not shown) other ribozyme and deoxyribozyme motifs are used.

FIG. 2 illustrates combined modular rational design and in vitro selection for FMN-sensitive allosteric ribozymes. (A) Tripartite construct consisting of a hammerhead ribozyme joined to an FMN-binding aptamer (boxed, SEQ ID NO: 3) via a random-sequence bridge composed of eight nucleotides (N). The three stems that form the unmodified ribozyme are designated I, II and III and the site of RNA cleavage is indicated by the arrowhead. The randomized bridge serves both as a partial replacement for stem II of the ribozyme and as a flanking stem for the aptamer. The G-C base pair immediately adjacent to the catalytic core is needed for the hammerhead ribozyme to achieve maximal catalytic activity (9,42). Selection for FMN-inducible (B) and FMN-inhibited (C) allosteric ribozymes gave rise to RNA populations that respond either positively or negatively to the presence of FMN, respectively. The initial RNA pool (G0) and successive RNA populations (G1 through G6) are identified.

FIG. 3 shows bridge sequences and kinetic parameters for individual allosteric ribozymes. (A) Sequences and corresponding ribozyme rate constants for eight classes of induction elements isolated from G6. Plotted for each class is the logarithm of the observed rate constant for self-cleavage in the absence (open circles) or presence (filled circles) of FMN. The base pairing schemes depicted for each bridge were generated by assuming that no base-pair shift relative to the G-C base pair remaining in stem II had occurred. Indicated are classes that display greater than 20% misfolding (*) and a class wherein an extraneous mutation exists in the stem-loop region of the aptamer domain (+). H1 is an unmodified hammerhead ribozyme (4,7,8) that displays maximum catalytic activity and that remains unaffected by the presence of FMN. (B) Fold-activation of catalytic activity (k_{obs}+/k_{obs}−) achieved in the presence of ligand for each class of FMN-inducible ribozyme. (C) Sequences and corresponding ribozyme rate constants for five classes of inhibition elements isolated from G6. Nucleotide deletions are represented as dashes. (D) Fold-inhibition of catalytic activity (k_{obs}+/k_{obs}−) achieved in the presence of ligand for each class of FMN-inhibited ribozyme.

FIG. 4 illustrates rapid ligand-dependent modulation of allosteric ribozymes. Tripartite ribozyme constructs carrying either a class I induction element (A) or a class II inhibition element (B) are depicted. Sequences for the aptamer and ribozyme domains are as shown in FIG. 2. The performance of these ribozymes in the presence and absence of FMN are evident from plots (C) and (D), which show the natural logarithm of the fraction ribozyme remaining un-cleaved versus time relative to FMN addition. Inset plots provide an expanded view of ribozyme responses to FMN addition.

FIG. 5 shows the proposed ‘slip-structure’ mechanism for allosteric regulation mediated by the class I induction element (A) and class II inhibition element (B) is illustrated. Shown are the proposed stem II secondary structures of the ligand-bound and unbound states of the FMN-modulated ribozymes. Not depicted are the left- and right-flanking sequences which comprise the aptamer and ribozyme domains, respectively. Asterisks denote the G and C residues of the hammerhead ribozyme that must pair to support catalysis, and the A and G residues of the FMN aptamer that become paired upon ligand binding. Also shown are bimolecular ribozyme constructs containing stem II elements designed to simulate the active or inactive slip structures proposed for the class I induction module (C; I-1 through I-3, SEQ ID NOs 4 to 6) or the class II inhibition module (D; II-1 and II-2, SEQ ID NO: 7). Thick lines identify nucleotides that form the bridge elements. Mutations made within 1-3 to reinforce the desired base-pairing conformation are encircled.

FIG. 6 illustrates modular characteristics of the class I induction element. (A) Sequence and secondary structures of allosteric ribozyme constructs containing either an FMN, theophylline, or ATP aptamer (constructs I(f), I(t), and I(a), respectively). The terminal A*G or G-C base pairs of each aptamer (denoted by asterisks) are interactions stabilized by ligand binding. (B) Qualitative assessment of the specificity of ligand-induced ribozyme self-cleavage. Internally ³²P-labeled constructs were incubated at 23° C. for 15 min in the absence (−) or presence of FMN (F; 200 μM), theophylline (T; 1 mM), or ATP (A; 1 mM). (C) Kinetic parameters k_{obs}− (open circles) and k_{obs}+ (filled circles) determined for each allosteric ribozyme construct in the absence or presence of its cognate ligand, respectively. (D) Allosteric activation of ribozyme function (k_{obs}+/k_{obs}−) is depicted for each construct.

FIG. 7. (A) Initial population (G0) for the in vitro selection of theophylline-sensitive allosteric hammerhead ribozymes. The theophylline aptamer (SEQ ID NO: 8) is appended to stem II of the hammerhead ribozymes through a random sequence region consisting of 10 nucleotides. N represents any nucleotide identity. The site of self-cleavage is indicated by the arrowhead. (B) In vitro selection and amplification of theophylline-activated allosteric hammerhead ribozymes. The fraction of each population that cleaves in the absence (open bars) or presence (filled bars) of theophylline is shown on the left axis, while the corresponding rate constant for self-cleavage is indicated on the right axis.

FIG. 8 illustrates the tripartite design for allosteric ribozyme construction like that shown in FIG. 1. (A) Sequence and secondary structure for an FMN-sensitive allosteric, ribozyme (66). In this construct, the cm+FMN1 communication module (boxed) separates the ribozyme and aptamer domains. This communication module (cm) is the first sequence class (1) that was previously identified to undergo allosteric activation (+) in the presence of flavin mononucleotide (FMN). Base-paired elements that are required for hammerhead ribozyme activity (I, II and III) are labeled according to Hertel, et al (72). An arrowhead identifies the site of hammerhead-mediated cleavage. (B) A tripartite construct carrying a randomized aptamer domain used as the pool to initiate in vitro selection. N₂₅ represents 25 nucleotides with random base identity.

FIG. 9 shows the allosteric selection scheme and the isolation of RNA sensors with new effector specificities. (A) Precursor RNAs are (I) subjected to negative selection in the absence of effector. Uncleaved RNAs are isolated by PAGE and subjected to positive selection in the presence of a mixture of the four cNMPs. Cleaved RNAs are (II) amplified by RT-PCR to generate double stranded DNA templates. The resulting DNAs are (III) transcribed using bacteriophage T7 RNA polymerase T7 RNAP) to generate a new population of RNA molecules that are (IV) subjected to the next round of negative and positive selections. (V) Double-stranded DNAs from the desired rounds of selection are cloned and sequenced for further analysis. The boxed T7 represents a double-stranded promoter sequence for T7 RNAP. (B) Emergence of ligand-specific allosteric ribozymes over the course of in vitro selection is reflected by plotting the ratio of cleavage yields (presence versus absence of effectors) for each round of selection (G1 through G28). Specificity of the ligand-sensitive populations that emerge throughout the selection are designated by the bars. Asterisk denotes a change in the selection protocol to avoid acidifying the RNA sample prior to initiating the positive selection reaction. Daggers identify the rounds of selection where the cNMP that functions as an effector in the previous round is added to the negative selection reaction in subsequent rounds. Line indicates a cleavage ratio of 1, which represents the value expected if the cleavage activity of the population as a whole were to exhibit no preference for the effector mixture. (C) Selective activation of RNA cleavage by cNMPs. Trace amounts of internally ³²P-labeled RNAs representing the populations G18′, G20′ and G23′ were incubated for 15 min in the reaction buffer used for in vitro selection (50 mM Tris-HCl, pH 7.5 at 23° C., and 20 mM MgCl₂) in the absence of effector (−) or in the presence of 500 (M of the 3′,5′-cyclic mononucleotides A, G, C and U as indicated. Reaction products were separated by denaturing 10% PAGE and the bands were visualized and quantified using a PhosphorImager and ImageQuant software (Molecular Dynamics). Open and filled arrowheads identify the precursor and 5′ cleavage products, respectively. The 3′ cleavage products have greater electrophoretic mobility than the significantly larger precursor RNAs and 5′-cleavage fragments, and therefore are not present on the images.

FIG. 10 shows allosteric modulation of hammerhead ribozymes by cNMPs. (A) Sequences of the original communication module domains (boxed) and the original random-sequence domains (N₂₅) for eight distinct clones isolated from the G18′ RNA population (SEQ ID NOs 9 to 16). Dashes within the N₂₅ domain represent nucleotide deletions that have occurred somewhere within this region. Numbers in parentheses report the number of identical clones with identical sequences. All isolates are identified as having effector-responsive allosteric function (+), show no response to the addition of effector (−), or the allosteric function was not determined (ND). Note that in nearly all cases, the communication module domains have acquired a minimum of one mutation. (B) Ligand-dependent cleavage of individual allosteric ribozymes isolated from the G18′ RNA population. RNA precursors (open arrowheads) produce greater amounts of Y-cleavage product (filled arrowheads) in the presence of 500 μM cGMP compared to its absence. The assays were conducted under in vitro selection conditions, and as a result, the product yields in the presence of effector versus the absence of effector reflect the advantage that each ribozyme maintains during the selective-amplification process. Reaction products were separated and visualized as described above in the legend to FIG. 9C. (C) The initial rate constants for the clones depicted in B in the presence (k_{obs}+, filled circles) or absence (k_{obs}−, open circles) of 500 μM effector are depicted on a log scale. These rate constants reveal “on/off” ratios that range between 5- and 510-fold under in vitro selection conditions. (D-F) Allosteric modulation of G20′ hammerhead ribozymes by cCMP (SEQ ID NOs 17 to 23). (G-1) Allosteric modulation of G23′ hammerhead ribozymes by cAMP (SEQ ID NOs 24 to 31). Details for the analysis of the cCMP- and cAMP-dependent ribozymes are as described in A-C.

FIG. 11 depicts information related to molecular recognition of cAMP by cAMP-3 RNA. (A) The caged cAMP analogue adenosine 3′,5′-cyclic monophosphate, P′-(2-nitrophenyl)ethyl ester is converted to 3′,5′-cAMP by brief irradiation with long wave UV light. (B) Allosteric activation of cAMP-3 RNA by uncaged cAMP. The plot depicts the natural logarithm of the fraction of precursor RNAs that remain uncleaved at different incubation times in the presence (squares and circles) or absence (triangles) of 2 mM caged cAMP. Shaded and filled symbols represent data collected during or after UV irradiation, respectively. Irradiated mixtures were exposed between t=3.5 and 4.5 min (dashed lines). The ribozyme is activated only when irradiated (filled symbols) in the presence of cAMP.

FIG. 12 provides data related to molecular recognition of cAMP by cAMP-1 RNA. (A) The effects of in situ depletion of cAMP from the reaction buffer prior to the addition of the cAMP-1 allosteric ribozyme were determined by using 3′,5′-cyclic nucleotide phosphodiesterase and calmodulin. Precursor RNAs (open arrowhead) undergo activation when incubated in reaction mixtures containing cAMP (+, lanes 3 and 4) or when incubated in reaction mixtures containing cAMP and including either phosphodiesterase (pho) or calmodulin (cal) (lanes 5 and 6, respectively). When combined, the phosphodiesterase and its activator calmodulin promote the hydrolysis of >90% of the cAMP to yield 5′-AMP during a 40 min preincubation (preinc) at 30° C. The cAMP-1 RNA, which does not accommodate 5′-AMP as an effector (see FIG. 13, below) is no longer activated under these conditions (lane 7). Reaction products were separated and visualized as described in the legend to FIG. 9C. (B) Plot depicting the activation of cAMP-1 by the addition of cAMP to 500 μM (indicated by the arrow) after exhaustive depletion of an original sample of cAMP. This reaction is derivative of that depicted in lane 7 of A, but where an 80 min preincubation with the phosphodiesterase/calmodulin mixture was used to more thoroughly deplete the initial input of cAMP. Filled and open circles identify data points collected before and after addition of the second aliquot of cAMP, respectively.

FIG. 13 shows patterns of selective molecular recognition by cNMP-dependent allosteric ribozymes. Each of the three allosteric ribozymes cGMP-1, cCMP-1 and cAMP-1 were incubated for 15 min under in vitro selection conditions in the absence of effector (−), in the presence of 500 μM of its cognate cNMP effector, or similarly with a panel of different effector analogues. Internally ³²P-labeled precursor RNAs and the resulting Y-cleavage fragments are identified by open and filled arrowheads, respectively. G, C and A represent the nucleosides guanosine, cytidine and adenosine, respectively. cIMP represents inosine 3′,5′-cyclic monophosphate. Reaction products were separated and visualized as described in the legend to FIG. 9C.

FIG. 14 shows rapid effector-mediated activation of allosteric ribozymes. Reactions containing internally ³²P-labeled precursor RNAs as indicated were incubated for a brief time in the absence of effector, then 5 mM of their corresponding effector was added (dashed line) and the reaction was continued. The x-axis reflects the time relative to the addition of effector. The precursor (open arrowheads) and resulting 5′-cleavage fragments (filled arrowheads) were separated, visualized and quantitated as described in the legend to FIG. 9C. The natural logarithm of the fraction of precursor remaining is plotted for each data point generated before (open circles) or after (filled circles) addition of effector, where the change in slope reflects the allosteric response of each ribozyme.

FIG. 15 graphs effector binding affinities and the dynamic ranges for various allosteric ribozymes. The logarithm of the rate constant for ribozyme cleavage versus the logarithm of the effector concentration is plotted for each of the ten clones depicted in FIG. 10. The minimum possible values for apparent KD for each clone is represented by the location of the shaded arrowhead on the x-axis of each plot (assuming that kobs at 10 mM effector reflects kₘₐₓ). The difference in rate constants that is brought about by progressively increasing the concentration of the effector reflects the dynamic range for each clone. For example, log k_{obs}, for cAMP-1 increases from −3 in the absence of effector (FIG. 31) to −0.5 upon saturation of effector. Variation in the rate constant brought about by different concentrations of effector corresponds to a dynamic range for cAMP-1 of ˜300 fold. Dashed lines reflect the concentration of effector (500 μM) used during in vitro selection.

FIG. 16 illustrates reactive DNA biochips prepared with highly selective multidomain polynucleotides of the invention in a grid assay. The indicated sensors were applied to the chips as indicated by arraying different ligand-sensitive sensors on a surface using standard nucleic acid immobilization techniques, and the chips are exposed to samples containing various potential effector molecules. Compounds responsive to sensors denoted B19, C3, G5, G9, P15, and S2 are found to be present in concentrations above the threshold level.

### DETAILED DESCRIPTION OF THE INVENTION

This invention is based upon the finding that combining a polynucleotide actuator domain and a receptor domain, with a bridging domain that provides communication between the two, results in precision polynucleotide sensors. By use of modular rational design strategies that mix and match domains, multidomain polynucleotides are modified to generate large numbers of structurally parallel sensors that are then screened to identify sensors displaying optimal binding and/or reporting activity.

In the practice of the invention, purified functional polynucleotides are generated or selected which comprise an actuator domain, a receptor domain, and a bridging domain such that a signalling agent such as binding of a ligand to the receptor domain triggers a conformational change in the bridging domain which modulates the activity of the actuator domain. The overall structure functions as a molecular switch, with the signalling agent turning the reporter domain partially or totally “on” or “off” upon interaction with the receptor domain which then communicates via the bridging domain. The molecular bridge in the engineered sensor is not passive, but is instead a functional communication module that activates, accelerates, decelerates, or triggers the action of the catalytic or reporter actuator. Indeed, as will be discussed in greater detail below, the invention encompasses methods for providing or enhancing allosteric properties in a polynucleotide by inserting into the polynucleotide communication module sequences that bridge receptor domains and actuator domains in the polynucleotide such that the sequence modulates the activity of the actuator domain when the receptor domain is acted upon by a ligand or a physical signal. In some embodiments, different communications modules are additionally used to modify the properties of the catalytic or reporter actuator, such as changing the kinetics of a reaction rate. In other embodiments, the bridging domain can overlap the receptor or reporter domain such that it is no longer present as a distinct structural entity. Novel allosteric polynucleotides of the invention are generated using modular rational design strategies by varying the actuator domain or the receptor domain and screening the sensors so produced to identify sensors having optimal sensing and/or reporting activities. The generation of some novel RNA sensors using this method is illustrated in Example 3 below.

Other additional domains may also be part of the construct such as, for example, multiple receptor domains for the measurement or detection of multiple components in a mixture tested by the sensor. Two or more domains may be partially or completely overlapping or non-overlapping, or contain both partially overlapping and non-overlapping sequences. Thus, as used herein, a “domain” is a functional designation, not a physical one, and sensors of the invention do not necessarily comprise different combinations of at least three distinct sequences directly or indirectly linked together, but instead can comprise sequences wherein some or all of the bases in the domains overlap with one another.

Multidomain polynucleotide molecular sensors of the invention may be RNA, RNA analogues, DNA, DNA analogues, or mixtures thereof. Analogues include chemically modified bases and unusual natural bases such as, but not limited to, 4-acetylcytidine, 5-(carboxyhydroxymethyl)uridine, 2′-O-methylcytidine, 5′-carboxymethylaminomethyl-2-thioridine, 5-carboxymethylaminomethyluridine, dihydrouridine, 2′-O-methylpseudouridine, β-D-galactosylqueosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methylguanosine, 2-methyladenosine, 3-methylcytidine, 5-methylcytidine, N6-methyladenosine, 7-methylguanosine, 5-methylaminomethyluridine, 5-methoxy-aminomethyl-2-thiouridine, β-D-mannosylqueosine, 5-methoxycarbonylmethyluridine, 5-methyloxyuridin, 2-methylthio-N-6-isopentenyladenosine, N((9-β-D-ribo-furanosyl-2-methylthiopurine-6-yl)carbamoyl)threonine, N-((9-β-D-ribofuranosyl-purine-6-yl)N-methyl-carbamoyl)threonine, uridine-5-oxyacetic acid methyl ester, uridine-5-oxyacetic acid, wybutoxosine, pseudouridine, queosine, 2-thiocytidine, 5-methyl-2-thiouridine, 2-thiouridine, 4-thiouridine, 5-methyluridine, N-((9-β-D-ribofuranosylpurine-6-yl)carbamoyl)threonine, 2′-O-methyl-5-methyluridine, 2′-0-methyluridine, wybutosine, and 3-(3-amino-3-carboxypropyl uridine. Further encompassed by the invention are polynucleotides modified during or after preparation of the sensor using standard means.

As summarized above, polynucleotide sensors of the invention are designed and constructed independently or together to comprise the actuator domain and receptor domain in communication with the bridging domain such that binding of a ligand to the receptor domain and/or a signal triggers a conformational change in the bridging domain which positively and/or negatively modulates the activity of the actuator domain. Where enzyme polynucleotides are employed, the reaction rate may be enhanced or inhibited by reversible binding to small effector molecules such as metal ions and/or compounds having a molecular weight of less than about 300. The effector molecule or effect binds to or affects a site that is spatially distinct from that of the enzyme or reporter domain, and rapidly interconvert from and “off” state to an “on” state, or vice versa, or intermediate states between “off” and “on”, reversibly, via the bridging domain on a time scale that is relevant for their use as biosensors (i.e., in preferably less than 60 minutes, even more preferably in less than 6 minutes, and in most cases in less than 1 minute, e.g., within seconds). Since they are responsive to ligands and/or signals, multidomain polynucleotides of the invention have a variety of uses, particularly as sensing elements in clinical, industrial, agricultural, and environmental analyses, and as genetic control or report elements for gene expression.

Sensors of the invention may be employed in solution or suspension or attached to a solid support. Alone or as a component of an analytical kit or probe, the polynucleotides are used to detect the presence or absence of a ligand or a signal in a sample by contact of the sample with the polynucleotide. In a typical practice of these methods, a sample is incubated with the polynucleotide or device comprising the polynucleotide as a sensing element for a time under conditions sufficient to observe the catalytic or reporter effect produced by the actuator domain. This is monitored using any method known to those skilled in the art, such as measurement and/or observation of polynucleotide self-cleavage or ligation; binding of a radioactive, fluorescent, or chromophoric tag; binding of a monoclonal or fusion phage antibody; or change in component concentration, spectrophotometric, or electrical properties. It is an advantage of the invention that current biosensor technology employing potentiometric electrodes, FETs, various probes, redox mediators, and the like can be adapted for use in conjunction with the new polynucleotide sensors of the invention for measurement of changes in polynucleotide function or configuration initiated by the actuator domain.

Sensors of the invention may be used to detect the presence or absence of a compound or other ligand, as well as its concentration. Sensors can be engineered to detect any type of ligand such as, but not limited to, all types of organic and inorganic compounds, metal ions, minerals, macromolecules, polymers, oils, microbial or cellular metabolites, blood or urine components, other bodily fluids obtained from biological samples, pesticides, herbicides, toxins, nonbiological materials, and combinations of any of these. Organic compounds include various biochemicals in addition to those mentioned above such as amino acids, peptides, polypeptides, nucleic acids, nucleosides, nucleotides, sugars, carbohydrates, polymers, and lipids. One or more ligands may be sensed by the same sensor in some embodiments.

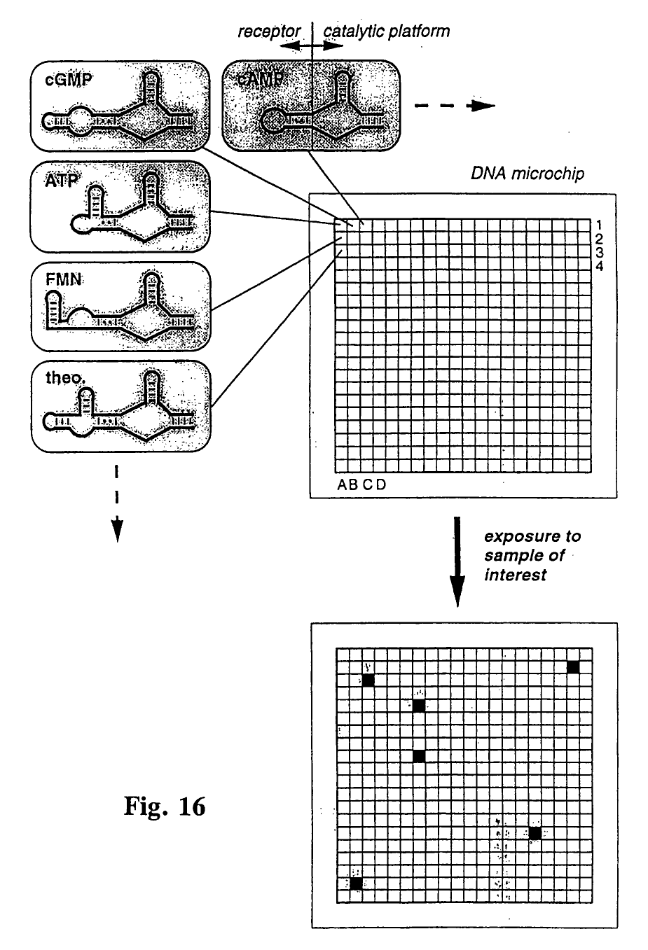

Thus, sensors of the invention have wide application in clinical diagnosis and medicine and veterinary medicine, including the determination of blood components such as glucose, electrolytes, metabolites and gases; serum analyte determinations; bacterial and viral analyses; pharmaceutical and drug analyses; drug design; cell recognition/histocompatibility; cell adhesion studies; bacterial and viral analysis; DNA probe design; gene identification; and hormone receptor binding. Industrial applications include the detection of vitamins and other ingredients, toxins, and microorganisms in foods; military applications such as dispstick testing; industrial effluent control; pollution control and monitoring; remote sensing; process control; separation chemistry; and biocomputing. Agricultural applications include farm and garden analyses and evaluations of genetic control and effects of compounds, particularly small molecules, in transgenic plants and animals (including in vivo measurements). Multiple sensors may be placed on a single sensory element or chip, such as that illustrated in FIG. 16, to detect multiple ligands and other signalling agents.

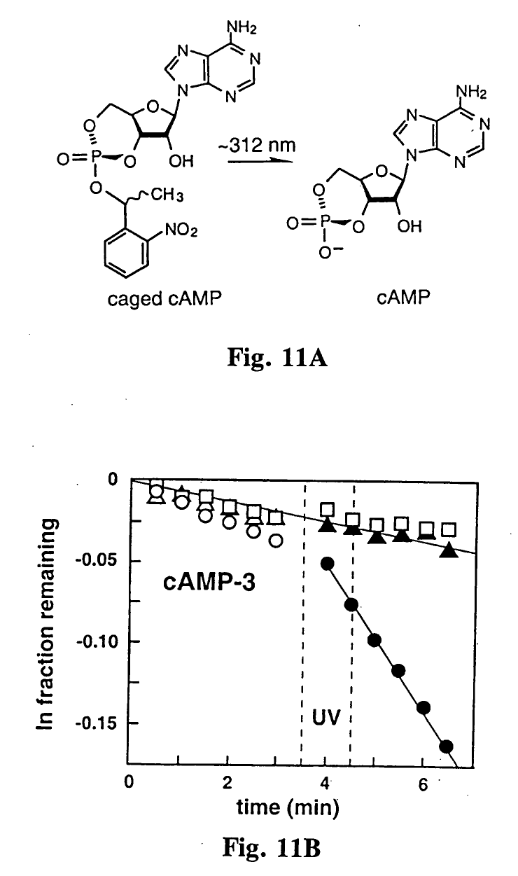

In alternate embodiments, or in combination with ligand detection, multidomain polynucleotide sensors of the invention can be engineered to respond to any change in energy reception measurable by a change in molecular conformation, a physical signal, an electromagnetic signal, and combinations thereof including, but not limited to radiation such as UV irradiation of caged effectors illustrated in FIG. 11, temperature changes, pH, ionic concentration, shock, sound, and combinations thereof.

Upon stimulation by a ligand or signal, the actuator domain modifies its catalytic function or reporter function. Any observation of a change in polynucleotide configuration or function may be employed to determine this. In many embodiments, an observation of a chemical reaction is made such as measurement and/or observation of polynucleotide self-cleavage or ligation, substrate cleavage, or generation of a catalytic reaction product using standard assays. In others, simple binding of a radioactive, fluorescent, or chromophoric tag, binding of a monoclonal or fusion phage antibody, or binding of a tagged antibody is observed. Alternatively, changes in component concentration, temperature, pH, appearance, spectrophotometric or electrical properties and the like, may be observed.

As mentioned above, the invention correspondingly provides methods for detecting one or more ligands and/or signals by contacting the sample with a polynucleotide sensor of the invention responsive to the ligand and/or signal. Use of sensors responsive to more than one ligand and/or signal, tandem use of an array of multiple sensors each responsive to different ligands and/or signals, and tandem use of multiple sensors with sensors responsive to more than one ligand and/or signal, in many cases attached to a solid support, are encompassed by the invention.

Multidomain polynucleotide sensors of the invention may also be used for the control and/or report of gene expression in vivo. For example, ribozymes exhibiting new allosteric binding specificity and refined kinetic characteristics are generated using allosteric selection are made to function inside cells with a level of catalytic performance that is of biological significance. In these embodiments, regulation or report of gene expression in a cell of an organism is achieved by operably linking a sensor to a genetic molecule in the cell such that the biological or phenotypic activity encoded by the gene is modulated in accordance with modulation of the activity of the actuator domain. RNA sensors may be inserted anywhere in the coding region of an mRNA encoding a gene-of-interest, or in close proximity thereto, or in the 5′-leader or 3′-tail regions, so long as the sensor functions to stimulate, terminate, or modulate expression of gene translation in the presence of the sensor's corresponding ligand(s) and/or signal(s). Likewise, DNA sensors may be inserted anywhere in a gene-of-interest or a gene regulating it, including in regions encoding gene self-destruction, regions upstream of gene expression, as well as in the coding regions of the gene, so long as the sensor functions to stimulate, terminate, or modulate gene transcription in the presence of the sensor's corresponding ligand(s) and/or signal(s).

Sensors are inserted in genetic molecules for control and/or report of gene expression using standard methods of introducing foreign genes into cells. The methodology depends upon the gene of interest, and typically includes cell transfection, transformation or transduction of cells using plasmids; Herpes, adeno, adeno-associated, vaccinia, retroviral, and other insertion vector viruses; and liposomes. Although less common, insertion of naked RNA (or DNA) by cleavage of cellular genetic material followed by ligation may also be employed.

Gene expression may be regulated or reported in any type of organisms, including microorganisms, plants, and animals. Gene regulation is achieved by administration to a cell having a sensor attached to a genetic molecule, the appropriate ligand(s) and/or signal(s) using standard methods. Administration of ligands to microorganisms, for example, is typically achieved simply by adding the ligand to the medium or removing it, or by perfusing the bacteria, yeast, or molds. Ligands may be administered to plants by spraying or injecting the plant itself, or applying it to the soil and/or with water. Ligands may be administered to animals orally, topically, intravenously, and intraperitoneally, typically in association with a pharmaceutically acceptable carrier. Report of gene expression is correspondingly determined by measurement of receptor binding to ligand, and can be used for non-invasive diagnostics of nearly any biological or pharmaceutical compound of interest administered to, or produced by, an organism. In this context, multidomain polynucleotides of the invention are useful both in non-invasive diagnostics as well as for control of therapeutic ribozymes.

The invention correspondingly provides processes for preparing polynucleotides that are responsive to the presence or absence of a chemical effector or other ligand, a physical signal, an electromagnetic signal, or combinations thereof, comprising linking an actuator domain, a receptor domain, and a bridging domain together such that binding of a ligand to the receptor domain and/or signal triggers a conformational change in the bridging domain which modulates the activity of the actuator domain. Other sensors can be developed by mixing and matching domains from different sensors.

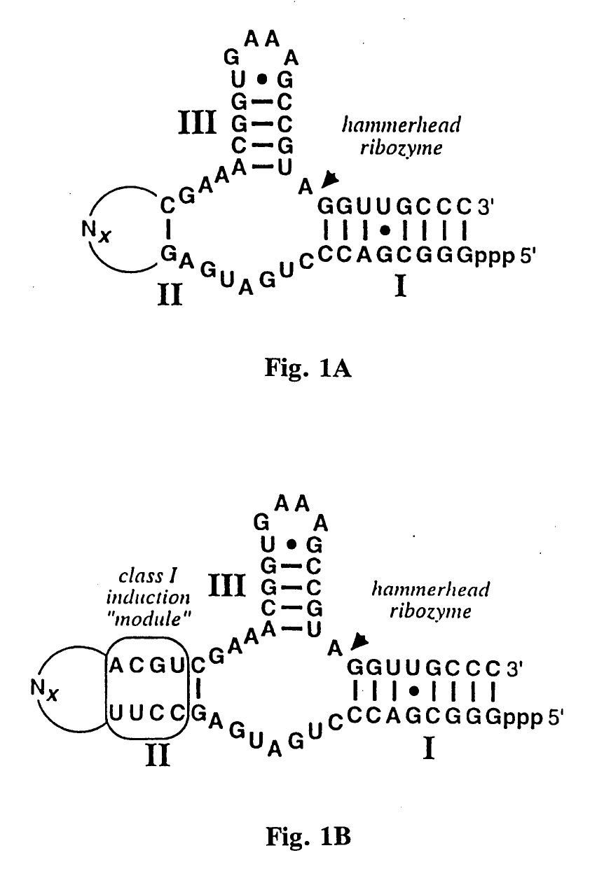

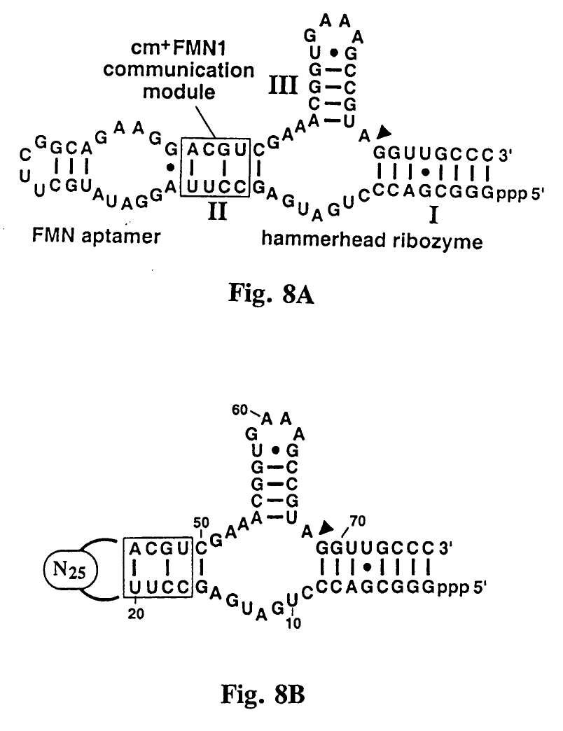

Some sensors of the invention are developed through allosteric selection. Allosteric selection is an in vitro selection technique for the development of allosteric nucleic acid enzymes that are controlled by ligands for which an aptamer has not previously been identified. In this capacity, allosteric selection also represents a novel approach to the generation of aptamers than bind target ligands. For this purpose, a random sequence library is typically appended to a catalytic nucleic acid motif such as the hammerhead ribozyme illustrated in FIGS. 1 and 8. The random domain may be attached directly to the ribozyme (FIG. 1A) or through an existing ‘communication modules’ (FIGS. 1B and 8). In the latter case, the communication module is expected to inhibit self-cleavage within the ribozyme domain in the absence of a target ligand. In this manner, in vitro selection for self-cleavage in the presence of target ligands will yield new aptamers and allosteric ribozymes if ligand binding to unique sequences derived from the random region triggers a conformational change that is conducive to ribozyme cleavage.

Using this selection strategy, four natural 3′,5′-cyclic mononucleotides including the second messengers cGMP and cAMP were targeted by hammerhead ribozymes in Example 3. This collection of molecules provides a diverse set of targets that are of biological importance and that challenge the structure formation and molecular recognition capabilities of RNA. Ribozymes that rapidly self-cleave only when incubated with their corresponding effector compounds were identified. Representative RNAs exhibit 5,000-fold activation in the presence of cGMP or cAMP, thus displaying precise molecular recognition characteristics and operating with catalytic rates that match those exhibited by unaltered ribozymes. These findings demonstrate that a vast number of ligand-responsive ribozymes with dynamic structural chacteristics can be generated in a massively parallel fashion. Moreover, optimized allosteric ribozymes provide especially selective sensors of chemical agents or as genetic control elements for the programmed destruction of cellular RNAs.

Allosteric selection of aptamers to small ligands has two distinct advantages over the conventional affinity chromatography methods for aptamer selection. First, aptamers to numerous ligands may be generated in a single selection rather than the laborious single ligand-single aptamer selection strategy afforded by affinity chromatography. Second, aptamers are selected to bind ligands free in solution rather than ligand that has been covalently modified and immobilized on a solid support. This aspect affords potential aptamers complete access to the entire ligand. It is conceivable that any effector-ribozyme pair could be developed using this approach. This unique process of nucleic acid development may therefore be used to develop nucleic acids that interact with a variety of ligands including small organic compounds, peptides or proteins, or other nucleic acids. In addition to ligand binding, allosteric selection also provides a means of developing nucleic acid motifs capable of detecting a variety of physical phenomena including pH, temperature, ionic conditions, or light.

While not wishing to be bound to any theory, it appears that the communication module function provided by the bridging domain is accomplished in sensors of the invention by one or a combination of mechanisms such as the ‘slip-structure’ interconversion set out in Example 1 below. Control can also be achieved using steric interactions such as binding of small compounds, structure stabilization such as unfolding or misfolding in the presence or absence of an effector, antisense effects based on simple nucleic acid base pairing, and/or quarternary structure. Any type of relay of a ligand-binding or physical or electromagnetic effect sensed by the receptor domain may be employed to transfer information to the actuator (reporter or catalytic) domain by the bridging domain.

It is an advantage of the invention that use of polynucleotides as sensors offer advantages over protein-based enzymes in a number of commercial and industrial processes. Problems such as protein stability, supply, substrate specificity and inflexible reaction conditions all limit the practical implementation of natural biocatalysts. DNA can be engineered to operate as a sensor under defined reactions conditions. Moreover, sensors made from DNA are expected to be much more stable and can be easily made by automated oligonucleotide synthesis. In addition, both DNA and RNA sensors may be selected for their ability to function on a solid support and are expected to retain their activity when immobilized.

As has been mentioned, the invention further encompasses the use of multidomain polynucleotide molecular sensors attached to a solid support for assays, diagnostics, catalytic processes, and the like. Immobilizing novel RNA or DNA enzymes provides a new form of coated surfaces for the efficient sensing of ligands or chemical transformations for testing of individual samples or in a continuous-flow reactor under both physiological and non-physiological conditions. The engineering of new sensors can be each tailor-made to efficiently respond to certain ligands or signals under user-defined conditions. Due to the high stability of the DNA phosphodiester bond, such surfaces when coated with multidomain DNA sensors are expected to remain active for much longer than similar surfaces that are be coated with protein enzymes or ribozymes.

A variety of different chromatographic resins and coupling methods can be employed to immobilize sensors of the invention on a support. For example, a simple non-covalent method that takes advantage of the strong binding affinity of streptavidin for biotin as previously described (45) may be employed. In other embodiments, sensors can be coupled to the column supports via covalent links to the matrix, thereby creating a longer-lived biosensor. Various parameters of the system including temperature, sample preparation, sensor size and sensitivity, and the like, can be adjusted to give optimal sensing properties. In fact, these parameters can be preset based on the kinetic or other characteristic displayed by the immobilized sensor.

In conclusion, the simultaneous use of rational and combinatorial approaches to enzyme engineering (41) provides a powerful approach to the design of new ribozymes and other sensors. As illustrated below, in some embodiments, tripartite ribozyme constructs generated using this strategy of polynucleotide engineering function as highly-specific sensors for various small organic compounds. A critical component of these constructs are the ligand-responsive bridge elements. These dynamic structural domains act as simple ‘communication modules’ that can be used to rapidly engineer new RNA molecular sensors simply by swapping domains within the context of the tripartite construct. In addition, the introduction of mutations into the receptor domain of the construct should make possible the in vitro selection of new ligand-binding domains based on the modulation of a catalytic or other reporter activity. In a similar manner, new RNA molecular sensors can be made that serve as new precision biosensors, or that function in vivo as genetic control or reporter elements that regulate gene expression in response to the presence of many different kinds of effector molecules.

### EXAMPLES

The following examples are presented to further illustrate and explain the present invention and should not be taken as limiting in any regard.

### Example 1

### Engineering Precision RNA Molecular Sensors

Ligand-specific molecular sensors composed of RNA were created by coupling pre-existing catalytic and receptor domains via novel structural bridges (65). Binding of ligand to the receptor triggers a conformational change within the bridge, and this structural reorganization dictates the activity of the adjoining ribozyme. The modular nature of these tripartite constructs makes possible the rapid construction of precision RNA molecular sensors that trigger only in the presence of their corresponding ligand.

Materials and Methods

Oligonucleotides. Synthetic DNA and the 14-nucleotide substrate RNA were prepared by standard solid phase methods and purified by denaturing (8 M urea) polyacrylamide gel electrophoresis (PAGE) as described previously (4). RNA substrate was 5′-³²P-labeled with T4 polynucleotide kinase and (γ-³²P)-ATP, and repurified by PAGE. Double-stranded DNA templates for in vitro transcription using T7 RNA polymerase were generated by extension of primer A (5′-TAATACGACTCACTATAGGGCGACCCTGATGAG, SEQ ID NO: 32)) on a DNA template complementary to the desired RNA. Extension reaction were conducted with reverse transcriptase (RT) as described previously (7).

In Vitro Selection. Selection for allosteric activation was performed by first preselecting each successive population (1 μM internally ³²P-labeled RNA; ref. 5) for self-cleavage without FMN in 10 μL reaction buffer (50 mM Tris-HCl (pH 7.5 at 23° C. and 20 mM MgCl₂) for 20 hr at 23° C. Preselections for G4-G6 were punctuated at 5 hr intervals by heating to 65° C. for 1 min to denature and refold any misfolded molecules. Uncleaved RNA was purified by denaturing (8 M urea) 10% (PAGE), eluted from excised gel, and precipitated with ethanol. The resulting RNA was selected by incubation in the reaction buffer in the presence of 200 μM FMN for the times indicated. Reaction times for positive selections during subsequent iterations of the selective-amplification process were decreased to favor allosteric ribozymes with the fastest rates of self-cleavage. Products separated by 10% PAGE were imaged and quantitated using a PhosphorImager and ImageQuaNT software (Molecular Dynamics). The 5′-cleavage fragments produced in the presence of FMN were isolated as described above, amplified by RT-PCR (primer A and primer B: 5′-GGGCAACCTACGGCTTTCACCGTTTCG (5,9, SEQ ID NO: 33), and the resulting double-stranded DNA was transcribed in vitro (5) to generate the next RNA population. Selection for FMN inhibition was conducted in an identical fashion, except that FMN was included in both the transcription and the preselection, but was excluded in the selection reaction, Individual molecules from G6 populations of both selections were isolated by cloning (TA Cloning Kit, Invitrogen) and analyzed by sequencing (ThermalSequenase Kit, Amersham).

Allosteric Ribozyme Assays. Reactions containing internally ³²P-labeled self-cleaving ribozyme (100 to 500 nM) and either 200 μM FMN, 1 mM theophylline, or 1 mM ATP were initiated by the addition of reaction buffer and incubated through several half lives with periodic sampling. Products were separated by denaturing PAGE and yields were quantitated as described above. Rate constants were derived by plotting the natural logarithm of the fraction of uncleaved RNA versus time and establishing the negative slope of the resulting line. The values for each rate constant given are the average of a minimum of three replicate assays, each that differed by less than two fold. Ribozymes carrying the class I induction element and the class II inhibition element were arbitrarily chosen for detailed analysis.

Bimolecular assays were conducted under single-turnover conditions with ribozyme (500 nM) in excess over trace amounts ˜5 nM of 5′-³²P-labeled substrate. Reactions were initiated by combining ribozyme and substrate that were preincubated separately for 10 min at 23° C. in reaction buffer. Kinetic parameters were generated as described above. Product yields were corrected for the amount of substrate that remained uncleaved after exhaustive incubation with the unmodified hammerhead ribozyme (5). The values for each rate constant given are the average of a minimum of two replicate assays that differed by less than two fold.

Results and Discussion

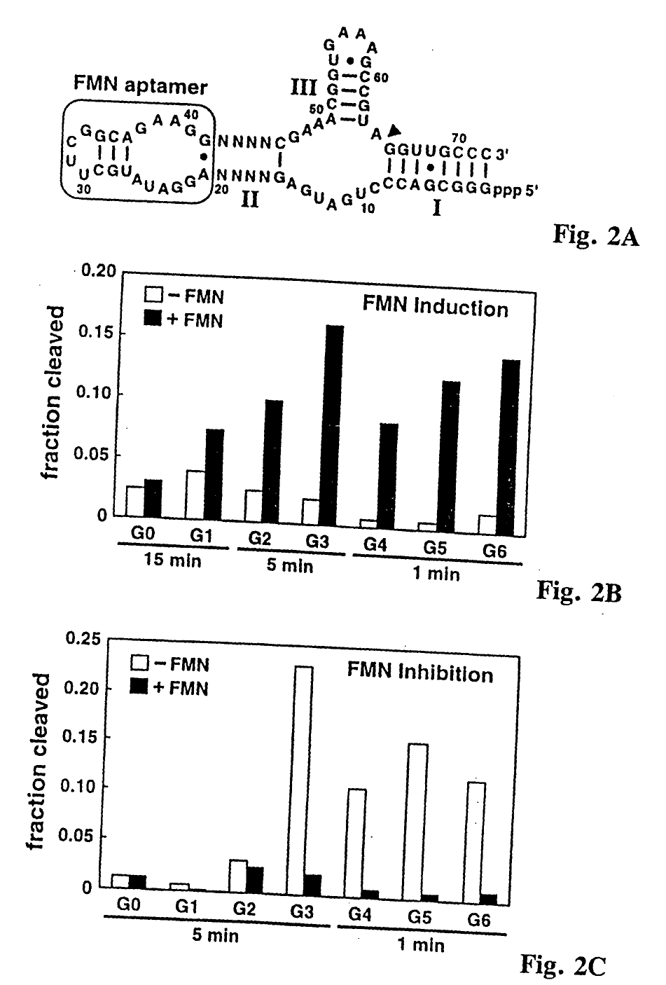

In Vitro Selection of Allosteric Ribozymes. A population of >65,000 variant RNAs composed of separate FMN-binding aptamer (26) and hammerhead ribozyme (27, 28) domains that are joined by a random-sequence bridge were generated (FIG. 2A). The bridge replaces a majority of the natural ‘stem II’ portion of the hammerhead motif—a structural element that is a critical determinant of ribozyme activity (29, 30). The randomized domain within the resulting tripartite construct will provide a sampling of alternative stem II elements that might respond to FMN binding in the adjacent aptamer domain, and confer either positive or negative allosteric control upon the adjoining ribozyme domain. Two identical RNA pools (˜6×10¹² molecules each) were subjected to in vitro selection (14, 15) either for FMN-dependent allosteric induction (FIG. 2B) or allosteric inhibition (FIG. 2C). To isolate bridges that direct the allosteric induction of ribozymes, a ‘negative selection’ for self-cleavage in the absence of FMN was applied to the first pool. RNAs that remained uncleaved during this reaction were isolated and subsequently subjected to a ‘positive selection’ for self-cleavage in the presence of FMN. This method is expected to favor the isolation of ribozymes that activate only when FMN is detected. In contrast, the second pool was both transcribed and pre-selected in the presence of FMN. The surviving RNA precursors were then subjected to positive selection in the absence of ligand, which favors the isolation of bridges that direct ribozymes to undergo allosteric inhibition.

Both RNA populations isolated after six rounds of selection (G6) display high sensitivity to FMN, demonstrating that the combined engineering approach is an effective means to generate ribozymes that function as highly-specific molecular switches. The in vitro selection process could have produced novel RNA structures that cleave by some other means under the permissive reaction conditions. For example, isoalloxazine rings like that found in FMN have been shown to promote photocleavage of RNA molecules (31) and could conceivably serve as a cofactor for a novel FMN-dependent ribozyme. However, the RNAs isolated by selection appear to cleave in a reaction that is solely mediated by the original hammerhead ribozyme domain that was integrated into each construct as determined by gel mobility of RNA cleavage fragments.

Sequence and Functional Characteristics of Isolated Bridge Elements.

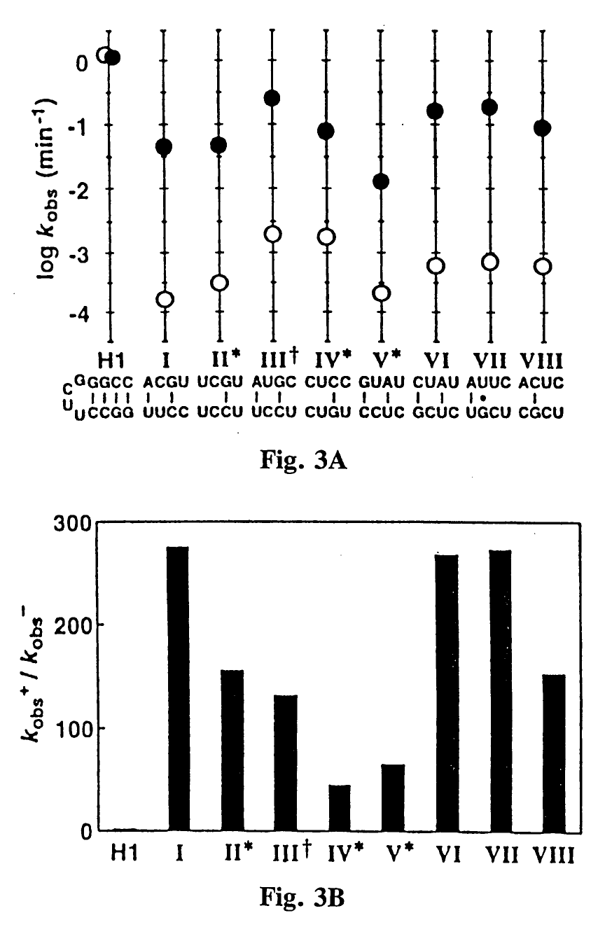

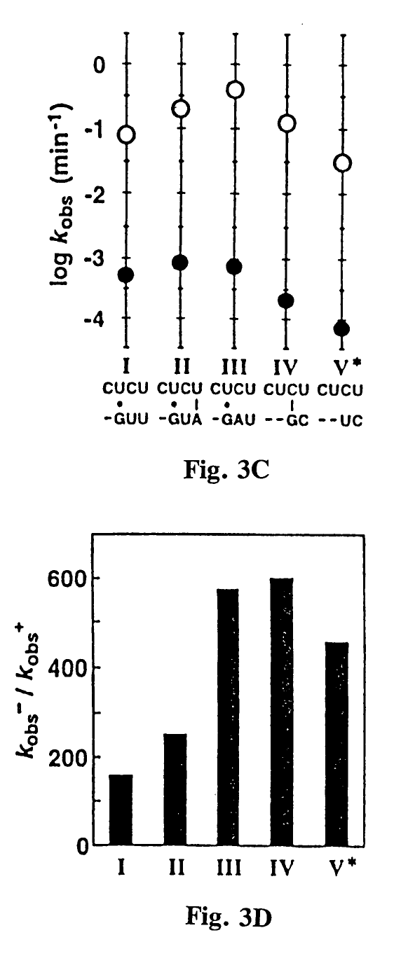

The G6 populations from both selections were cloned, sequenced, and assayed for allosteric function (FIG. 3). Eight distinct classes of bridges, designated as ‘induction elements’ I through VIII, were identified in the FMN-inducible RNA population. Ribozymes with these different classes of induction elements show unique rate constants for self-cleavage in the absence (k_{obs}−) or presence (k_{obs}+) of ligand (FIG. 2A). Most classes exhibit greater than 100-fold allosteric activation (k_{obs}+/k_{obs}−), with classes I, III, and VII exhibiting FMN-dependent rate enhancements of ˜270 fold (FIG. 2B). This allosteric induction is similar in magnitude to the kinetic modulation seen with some natural allosteric protein enzymes (32). Furthermore, the k_{obs}+ values attained by nearly all classes approach the maximum kobs (1.1 min−1) measured for an unmodified hammerhead ribozyme (FIG. 3A).

Likewise, five distinct classes of bridges were identified and were designated as ‘inhibition elements’ I through V (FIG. 3C). Unlike the FMN-inducible populations which showed an immediate response to in vitro selection, ligand-dependent inhibition of ribozyme function was not detected until G3 of this parallel selection. Interestingly, each of the five classes carries a 1- or 2-nucleotide deletion within the randomized bridge domain, suggesting that none of the sequence variants comprising the original RNA pool formed an adequate ligand-responsive element that could confer allosteric inhibition. The relative delay in deriving an FMN-inhibited RNA population may have been due to the necessary emergence of specific nucleotide deletions within the bridge domain—an occurrence that is dependent on the frequency of deletion events during the selective-amplification process. Consistent with this hypothesis is the fact that sequences of the inhibition elements are highly homologous, indicating that the emergence and diversification of a single responsive bridge domain may have given rise to all classes examined. All five classes demonstrate substantial allosteric inhibition (200 to 600 fold) in the presence of FMN (FIG. 3D).

Many of the bridge elements isolated by selection display maximum rate enhancements that are at least 10-fold lower than that measured for the unmodified hammerhead ribozyme H1 (FIG. 3). Among the allosteric ribozymes that display the largest rate constants for RNA cleavage carry the class III induction element (k_{obs}+=0.25 min⁻¹) or the class III inhibition element (k_{obs}−=0.45 min⁻¹). The maximum rate constants for these two ribozymes are, respectively, only four and two-fold slower than H1. Using similar in vitro selection methods, a population of theophylline-dependent ribozymes that use a tripartite configuration like that described for the FMN-sensitive RNAs was isolated. Individual theophylline-sensitive ribozymes from this population display rate constants that exceed 1 min⁻¹, thereby confirming that allosteric hammerhead ribozymes indeed can be made to operate as efficiently as the unmodified ribozyme.

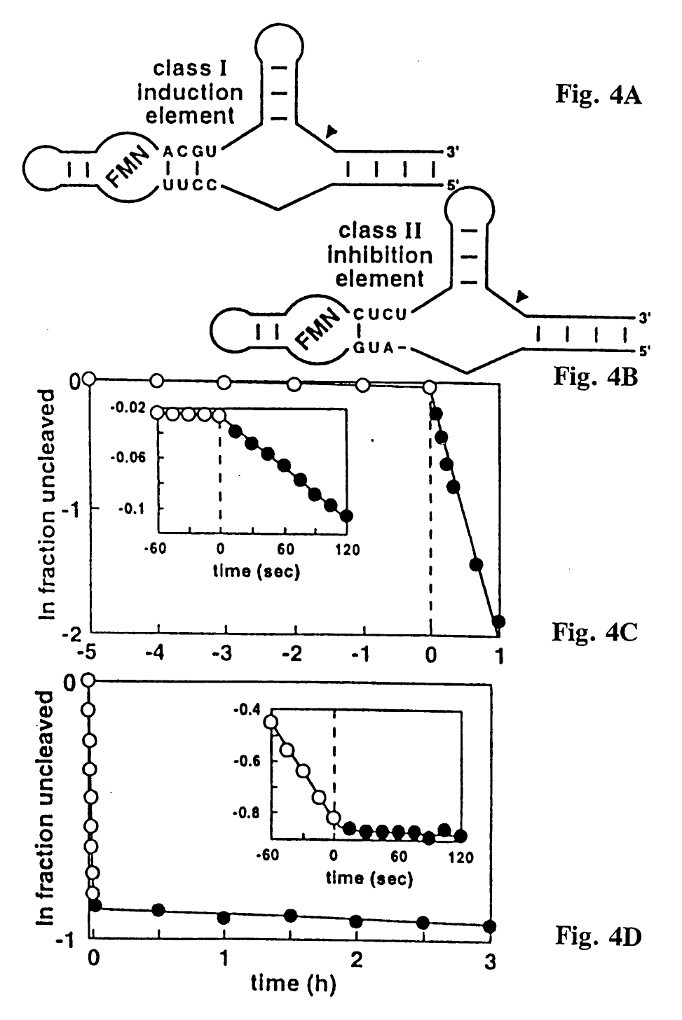

Rapid Interconversion Between Active and Inactive Ribozyme Structures. The inactive state for ribozymes that carry the class I induction element (FIG. 4A) is maintained for long periods of time in the absence of FMN, yielding only ˜ 1% self-cleavage per hour (FIG. 4C). However, self-cleavage is triggered almost instantaneously upon the addition of ligand (FIG. 4C; inset), in this case bringing about a 270-fold increase in catalytic rate. Presumably, the ‘off’ state maintained by induction elements in the absence of FMN lacks the ability to form the stable stem II structure that is necessary for ribozyme activity. Alternatively, each element forms a distinct structure that prevents formation of this essential stem. FMN binding establishes the ‘on’ state by inducing a conformational change in the aptamer that rapidly converts the induction element into a structure that is compatible with ribozyme function. In contrast, ribozymes that carry the class II inhibition element (FIG. 4B) rapidly self-cleave in the absence of FMN, but quickly convert to an inactive state upon addition of ligand (FIG. 4D; inset). Here, inhibition elements maintain the ‘off’ state by binding FMN and stabilizing specific bridge structures that preclude ribozyme function. Release of the ligand results in structural reorganization of the bridge and establishes the ‘on’ state of the adjoining ribozyme. However, it remains unclear what structural state is responsible for the slow rate of cleavage seen with the class II inhibition element when FMN is present. Further experimentation is needed to determine whether the FMN-ribozyme complex remains weakly active, or whether the small number of FMN-free RNAs present under equilibrium binding conditions solely contribute to the RNA cleavage rate that is observed.

Mechanism for Allosteric Function. The rapid ligand-dependent activation or inhibition of ribozyme function indicates that the conformational changes required to modulate activity must be highly responsive to ligand binding. It appears that for some elements this allosteric transition is achieved through localized base-pairing changes within each bridge domain, and that binding energy derived from ligand-aptamer complex formation is used to create this shift in structural configuration.

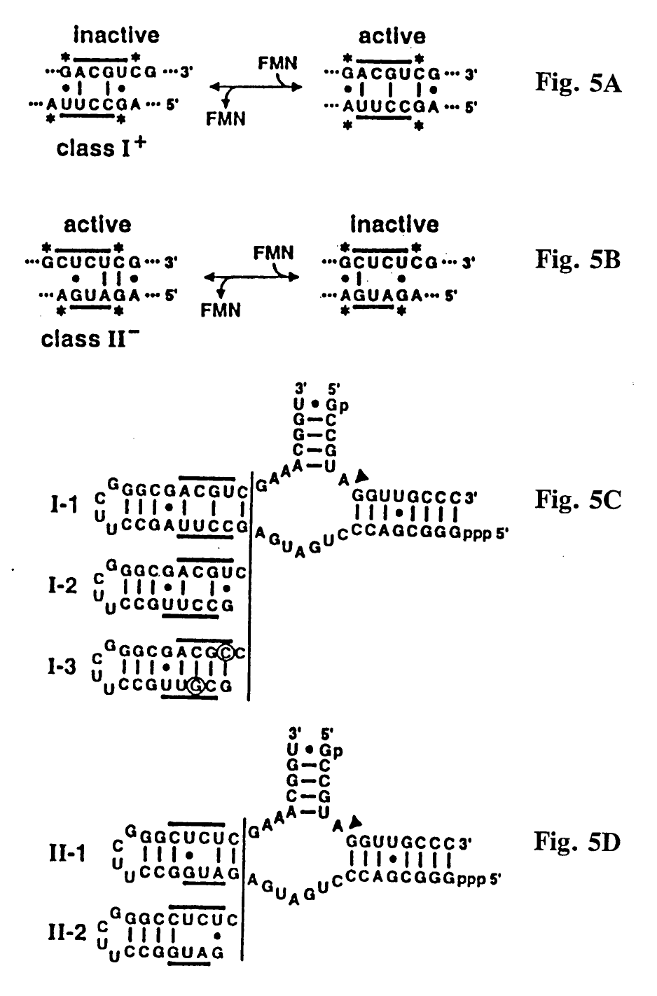

A critical component of the proposed mechanism for both allosteric induction and inhibition is a single sheared A•G base pair, located within the aptamer domain immediately adjacent to the bridge, which forms only when FMN is bound (33, 34). With class I induction elements, the presence of FMN stabilizes the A•G base pair which in turn establishes a specific register for base pairing within the bridge (FIG. 5A). In the absence of this FMN-dependent structural constraint, base pairing throughout the bridge may ‘slip’ one base pair relative to the A•G interaction, thereby displacing the G-C base pair needed for ribozyme function. This inactive conformation would be maintained if no single nucleotide is bulged from the top strand of the bridge. Symmetric internal bulges are known to be more stable than asymmetric or single-nucleotide bulges (35). Therefore, the register that is set by the sheared A•G base pair may be faithfully propagated along the bridge element if the presence of symmetric internal bulges favor a continuously-stacked stem II domain. Interestingly, all inhibition modules acquired deletions that appear to be essential for their function. This corresponds well with a slip-structure mechanism, as a continuously-stacked bridge in this case would disrupt the critical G-C base pair of the ribozyme when FMN was bound, while the absence of FMN would allow proper ribozyme folding (FIG. 5B).

To further investigate this ‘slip structure’ mechanism for allosteric regulation, several ribozyme constructs were created using stable stem-loop structures in place of the FMN-binding domain (FIG. 5C). In its occupied state, the FMN aptamer forms a compact, approximately A-form RNA structure (34). Therefore, the stem-loop structures integrated into the test constructs should simulate the FMN-bound aptamer and enforce the putative slip structures necessary to either induce or inhibit ribozyme function. For example, construct I-1 is designed to simulate the structure of a class I induction element bound to FMN by enforcing the formation of the sheared A•G pair. Indeed, the k_{obs}, for I-1 in the absence of FMN is identical to the rate constant for the FMN-induced form of the parent allosteric ribozyme (Table 1). Two additional constructs (I-2 and I-3) were used to determine the rate constants when the opposing ‘slipped’ version is enforced with progressively stronger base pairing. Construct I-2 is not significantly inhibited when the aptamer is replaced by structures that should favor the inactive conformation. Perhaps in this context, a single bulged nucleotide along the top strand of the bridge may occur which would restore proper ribozyme folding. However, the activity of the adjoining ribozyme is substantially diminished when potential bulge formation is precluded by the introduction of additional base pairs in the bridge that forms construct I-3, consistent with the proposed mechanism for allosteric function.

Further evidence for a slip-structure mechanism was provided by examining the class II inhibition element. Here, FMN binding enforces a base pairing pattern that precludes formation of the active ribozyme conformation (FIG. 5D). In the absence of FMN, the loss of the A•G base pair may permit the remaining base pairs to slip by one nucleotide, thereby forming the active ribozyme conformation. Constructs II-1 and II-2, designed with stem-loop structures that enforce the two different base-pairing conformers, display rate constants that correspond closely with the values for the active and inactive states of the parent allosteric ribozyme, respectively (Table 1). In all examples, the bridge elements contain unpaired bases that presumably destabilize the stem structures and allow rapid interconversion between different structural states. A similar RNA switch mechanism may serve an important role in the structure and function of 16S ribosomal RNA (35, 36), a finding that indicates this mechanism for allosteric function may not be unprecedented. Although alternative mechanisms for allosteric function may be in operation, these striking correlations all are consistent with the proposed slip-structure mechanism. Similar studies with the remaining classes of bridge elements might reveal whether this mechanism is also more general in occurrence.

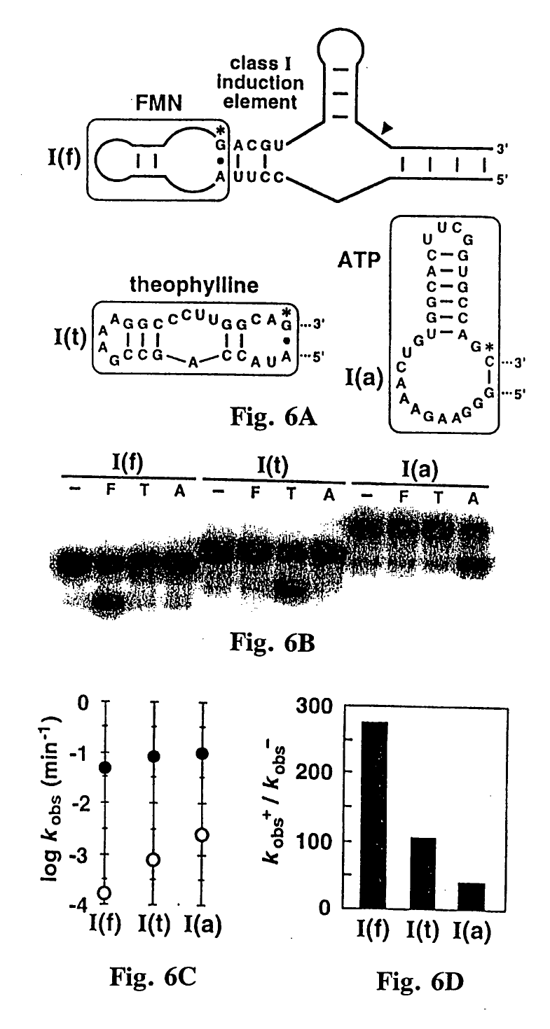

Engineering Allosteric Ribozymes with New Ligand Specificities. If binding energy derived from the ligand-aptamer complex is used to shift the thermodynamic balance between two slip-structure conformations, then each bridge may act as a generic reporter of the occupation state of the adjoining aptamer domain in a manner that is independent of the sequence and ligand specificity of the aptamer. To examine this possibility, the FMN aptamer was removed from the class I induction element of an FMN-sensitive ribozyme and replaced with either an aptamer that binds theophylline (37) or an aptamer that binds ATP (38) (FIG. 6A). In each case, ligand binding is known to stabilize base pairing of the terminal nucleotides of the appended aptamer (33, 38, 39). Therefore, adaptive binding of ligand by the aptamer may trigger the allosteric transition necessary for class I function. Indeed, each ribozyme construct undergoes self-cleavage only in the presence of its cognate ligand (FIG. 6B). Kinetic analyses (FIGS. 6C and 6D) show that the activity of the FMN-inducible ribozyme increases 270-fold in the presence of FMN, while the theophylline- and ATP-inducible ribozymes are activated 110- and 40-fold, respectively, only by their corresponding ligands. These findings indicate that the task of regulating ribozyme activity rests mainly on the bridge element, which relays information concerning the binding state of the aptamer to the adjoining ribozyme domain.

Although the class I induction element can be engineered to respond to several unrelated effector molecules, this characteristic is not universally applicable. For example, appending an aptamer for arginine (40) to the class I induction element failed to produce a significant allosteric effect. Two of three other classes of induction elements tested (classes VI and VII) also display modularity when engineered to carry the theophylline aptamer. However, class III induction element and class III inhibition elements showed no response to the addition of effector when similarly appended to the same aptamer. These findings indicate that the successful design of an allosteric ribozyme using this modular approach requires the fusion of compatible ‘matched pairs’ of aptamer and bridge domains.

TABLE 1
Catalytic rate constants for the ‘on’ and ‘off’ states of class I
(induction) and class II (inhibition) ribozymes compared to constructs
designed to simulate these states.

| Allosteric | k_{obs} (×10⁻¹ min⁻¹) | | Simulant | k_{obs} (×10⁻¹ min⁻¹) | |
|---|---|---|---|---|---|
| Ribozyme | ‘on’ | ‘off’ | Construct | ‘on’ | ‘off’ |
| Class I (induction) | 0.46 | 0.0017 | I-1 | 0.46 | — |
| | | | I-2 | — | 0.21 |
| | | | I-3 | — | 0.04 |
| Class II (inhibition) | 2.0 | 0.0080 | II-1 | 0.47 | — |
| | | | II-2 | — | 0.0020 |

### Example 2

In Vitro Selection of Theophylline-Sensitive Allosteric Hammerhead Ribozymes

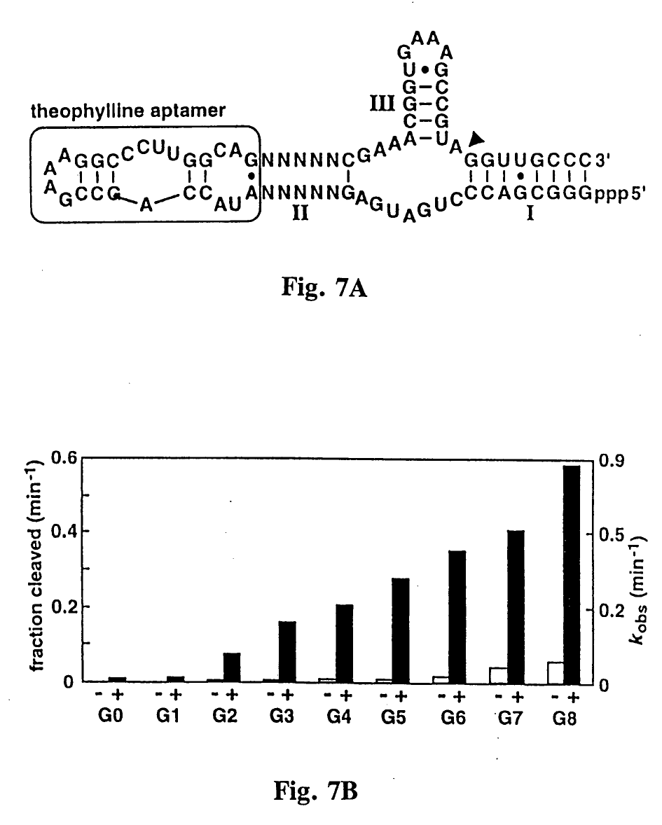

To investigate whether the process of developing communication modules may be applicable toward any number of aptamer-ribozyme combinations, in vitro selection for allosteric hammerhead ribozymes activated by theophylline binding has been performed. This selection has sought not only to validate the combined modular rational design and in vitro selection process, but develop new communication modules that try the limits of nucleic acid allostery. The initial population for the development of allosteric theophylline-sensitive ribozymes is conceptually identical to that previously demonstrated to yield FMN-sensitive catalysts. However, the theophylline aptamer was appended to stem II of the hammerhead ribozyme through a random-sequence region consisting of 5+5 or 10 total nucleotide positions (FIG. 7A). An RNA population resulting from eight rounds of in vitro selection and amplification of theophylline-activated ribozymes exhibits a marked capability to catalyze the self-cleavage reaction in the presence versus the absence of theophylline (G8; FIG. 7B). The population as a whole demonstrates an observed rate constant in the presence of ligand (k_{obs}+) that is essentially identical to the observed rate constant for an unmodified hammerhead ribozyme (˜1 min⁻¹). A number of individuals from the final population were isolated and further characterized to establish the sequences of the communication modules and the kinetic parameters for ligand-activated catalysis (Table 2). Many

TABLE 2

Communication module sequences and kinetic parameters of
theophylline-sensitive allosteric hammerhead ribozymes
isolated by in vitro selection.

| | | | | |
|---|---|---|---|---|
| clone | sequence^{a} | k_{obs}⁻(min⁻¹)^{b} | k_{obs}⁺(min⁻¹)^{c} | fold activation^{a} |

| | | | | |
|---|---|---|---|---|
| 5 | AUUGA | 4.3 × 10⁻⁴ | 1.1 | 2600 |
| | ·\|\| | | | |
| | GGACC | | | |
| 7 | UCGCU | 1.8 × 10⁻³ | 5.9 × 10⁻¹ | 330 |
| | ·\|\|\| | | | |
| | CCGCG | | | |
| 11 | UUUGA | 1.4 × 10⁻⁴ | 9.0 × 10⁻¹ | 6400 |
| | ·\|\|\| | | | |
| | GAACC | | | |
| 13 | UCAUA | 1.1 × 10⁻³ | 6.3 × 10⁻¹ | 570 |
| | ·\|\| \| | | | |
| | GGUCU | | | |
| 15 | UCUUA | 4.1 × 10⁻⁴ | 5.3 × 10⁻¹ | 1300 |
| | ·\| | | | |
| | GGCUC | | | |
| 16 | UCAUA | 2.6 × 10⁻⁴ | 9.3 × 10⁻¹ | 3600 |
| | ·\|\| | | | |
| | GGUCC | | | |
| 18 | UUAGA | 6.4 × 10⁻⁴ | 1.4 | 2200 |
| | ··\|\| | | | |
| | GGUCC | | | |

^{a}Sequence of each clone derived from nucleotides comprising the random region of the initial population.
^{b}Observed rate constant for self-cleavage in the absence of theophylline.
^{c}Initial observed rate constant for self-cleavage in the presence of 200 μM theophylline.
^{d}k_{obs}+/k_{obs}⁻.

isolates were demonstrated to achieve theophylline-dependent rate constants that approach or exceed 1 min⁻¹, where allosteric activation ranged from several hundred- to several thousand-fold. In this manner, selection for theophylline-sensitive allosteric hammerhead ribozymes has provided functionally superior catalysts without compromising the catalytic efficiency of the ribozyme motif. The use of combined modular rational design and in vitro selection techniques for the development of ligand-sensitive allosteric ribozymes is thus be widely applicable toward the development of novel allosteric catalysts.

### Example 3

Allosteric Selection of Ribozymes Responsive to cGMP and cAMP Messengers

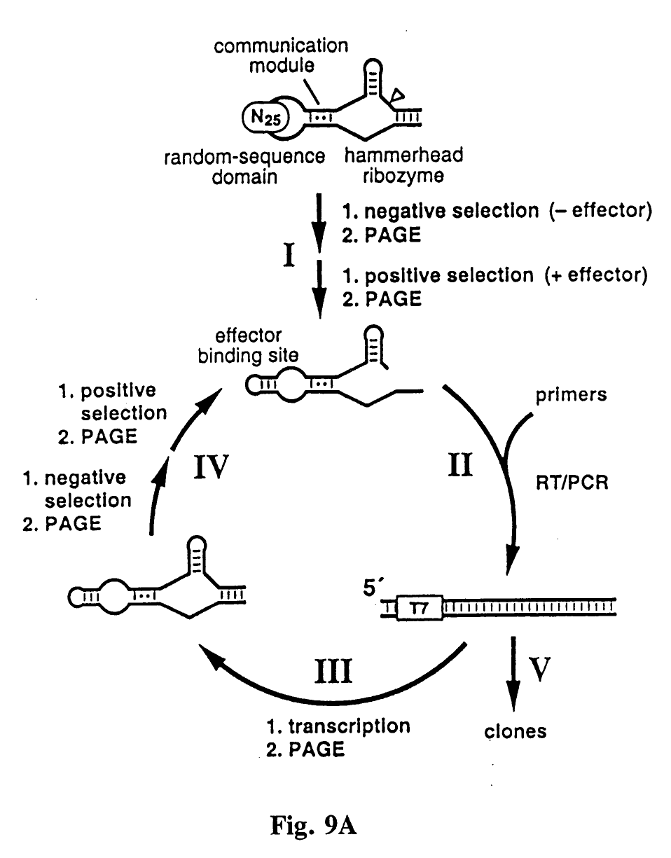

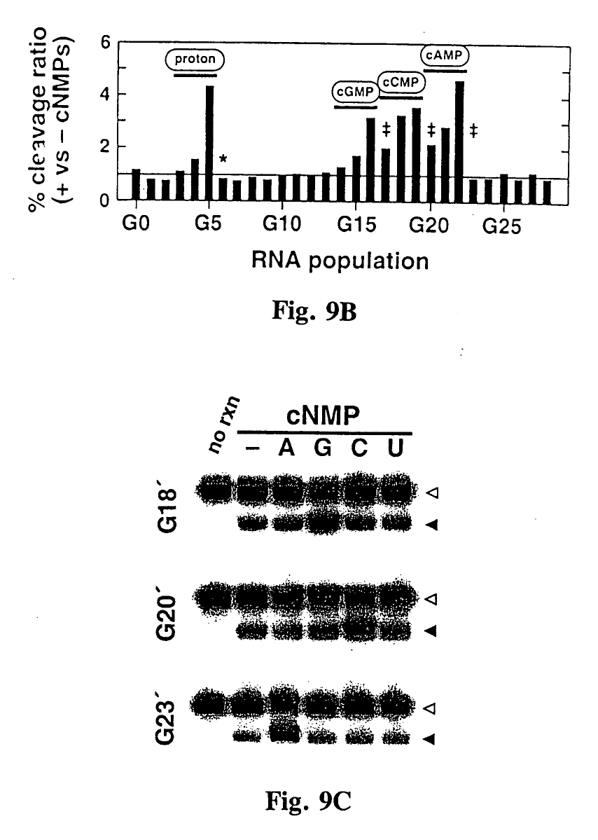

Example 1 illustrated the generation of a series of allosteric ribozymes using a three-domain construct (FIGS. 1 and 8). For several of the bridging domains identified, it was observed during the course of experiments that replacing the original aptamer domain with different aptamer domains having various ligand specificities produced new allosteric ribozymes with the corresponding effector dependencies. In other words, certain bridging domains or communication modules including the class I communication module (cm+FMN1) depicted in FIG. 8 appear to serve as generic reporters of the occupation state of different appended aptamers regardless of the particular ligand specificity. This example reports further studies conducted to investigate whether undiscovered aptamers could trigger ribozyme function if they were judiciously integrated into the effector-binding site of the tripartite RNA construct. A new construct was generated in which the entire effector-binding site is replaced with a 25-nucleotide domain comprised of random sequence (FIG. 8). The organization of this RNA construct facilitated the isolation of allosteric ribozymes with novel effector specificities using a selective-amplification process herein termed “allosteric selection” (FIG. 9A). This process favors the enrichment of the RNA population for those ribozymes that remain inactive in the absence of effector, but that are activated upon effector addition (73).

Materials and Methods

RNA Pool Preparation. DNA templates for the RNA pool depicted in FIG. 1B and the oligonucleotides used for RT-PCR were prepared by automated DNA synthesis (Keck Biotechnology Resource Laboratory, Yale University). All DNAs were purified by denaturing (8 M urea) polyacrylamide gel electrophoresis (PAGE) before use. The DNA template 5′-GGGCAACCTACGGCTTTCACCGT TTCGACGT(N₂₅)AAGGCTCATCAGGGTCGCC (4.15 nmoles, SEQ ID NO: 32+ACGT and SEQ ID NO: 34) was made double-stranded by extension in the presence of primer 2′ (5′-TAATACGACTCACTATAGGGCGACCCTGATGAG, 8.3 nmoles, SEQ ID NO: 32), which introduces the promoter for T7 RNA polymerase (T7 RNAP). The DNA extension reaction (300 μl) was carried out using SuperScript II reverse transcriptase (RT, GibcoBRL) according to the manufacturer's directions.

The resulting double-stranded DNAs were recovered by precipitation with ethanol and resuspended in a 2 ml transcription mixture containing 50 mM Tris-HCl (pH 7.5 at 23° C.), 15 mM MgCl₂, 5 mM dithiothreitol, 2 mM spermidine, 2 mM each of the four dNTPs, 200 μCi (³²P)UTP, and 60,000 U T7 RNAP. The transcription mixture was incubated at 37° C. for 1 hr and the resulting uncleaved precursor RNAs (internally ³²P-labeled) were isolated by denaturing 10% PAGE. Note that PAGE purification eliminates ribozymes that have undergone self-cleavage during the in vitro transcription reaction. This inherently introduces an additional negative selection step that disfavors the isolation of ribozymes, that function without activation by an effector. Moreover, this step disfavors the isolation of allosteric ribozymes that cannot distinguish between the intended cNMP target effectors and the NTPs that are required for in vitro transcription.

Allosteric Selection. In vitro selection for allosteric ribozymes that respond to the cNMPs (Sigma) was carried out using repeated rounds of negative and positive selection. For the first round of negative selection, an initial pool of RNA precursors (9.3 nmol, 5.6×10¹⁵ molecules) was incubated at 23° C. for 5 hr in a reaction mixture (930 μl) containing 50 mM Tris-HCl (pH 7.5) and 20 mM MgCl₂ in the absence of the four cNMPs. Precursor RNAs that resist cleavage during this incubation were isolated by denaturing 10% PAGE. Purified precursor RNAs were then subjected to the first round of positive selection at 23° C. for 30 min in the same reaction buffer (930 μl) containing 500 μM each of the four cNMPs. At this stage, cleaved products were purified by denaturing 10% PAGE and the 5′ cleavage fragments were recovered from the gel by crush-soak elution and amplified by reverse transcription followed by PCR (RT-PCR). Reverse transcription was conducted in a reaction buffer (400 μl total) using SuperScript II RT according to the manufacturer's directions cDNA and using primer 1 (5′-GGGCAACCTACGGCTTTCACCGTTTCG, SEQ ID NO: 33). Subsequent PCR amplification of the resulting cDNA using primers 1 and 2 (500 pmoles each) was conducted in a reaction mixture (2 ml total) containing 10 mM Tris-HCl (pH 8.3 at 23° C.), 50 mM KCl, 1.5 mM MgCl₂, 0.01% gelatin, 0.2 mM each dNTP and 50 U Taq polymerase (Promega). The reaction was thermocycled for the desired number of iterations at 94° C. for 30 sec, 55° C. for 30 sec, and 72° C. for 60 sec.

Additional rounds of selective amplification were repeated in a similar fashion using 15 min positive selection reactions until effector-sensitive ribozyme function was detected. Subsequent rounds of selection included both negative and positive selection steps that were conducted as described above using smaller RNA pools and with the reaction sizes scaled down accordingly. For the first 5 rounds of selection, a 10× stock mixture of cNMPs was added to the RNA pool prior to the addition of the remaining components of the reaction buffer. In subsequent rounds, the cNMP mixture was added after the reaction buffer to preclude the isolation of acid-sensitive ribozymes. In addition, negative selections were altered to more aggressively select against ribozymes that cleave slowly or that distribute between active and inactive conformations upon refolding. To disfavor slow-cleaving ribozymes, the negative selection time was increased from 5 hr to as much as 48 hr and multiple negative selection steps were occasionally employed prior to conducting positive selection. To disfavor misfolding ribozymes, periodic thermocycling was employed as described previously (65), or chemical denaturation with urea or mild alkali were used in an iterative fashion between periods of negative selection to induce multiple cycles of denaturation, renaturation and self-cleavage. Interestingly, ribozymes that use a misfolding strategy for survival also resisted the negative selection strategies that rely on thermal and urea-mediated denaturation (unpublished observations). Therefore, the use of alkaline denaturation proved most effective for negative selection.

Allosteric Ribozyme Characterization. RNA populations displaying cNMP-dependent self-cleavage were cloned (TOPO TA Cloning Kit, Invitrogen), sequenced (Thermo Sequenase Cycle Sequencing Kit, USB) and further analyzed by establishing the effector-mediated modulation of ribozyme kinetics.

Double-stranded DNA templates for individual allosteric ribozyme clones were prepared either by PCR amplification of the plasmid DNA using primers 1 and 2, or by preparation of the appropriate synthetic DNA template. Internally ³²P labeled RNAs were prepared by in vitro transcription as described above.

Initial rate constants for RNA self-cleavage were established by incubating trace amounts (˜100 nM) of internally ³²P labeled RNA precursors in selection buffer containing different concentrations of cNMP effectors as indicated for each experiment. Reactions were terminated by the addition of 2× PAGE loading buffer containing additional EDTA to sequester the Mg²⁺ cofactor (65). For each clone, a plot of the fraction of precursor cleaved (<20% processed) versus time gave a straight line where the slope reflects the initial rate constant for the ribozyme under the particular reaction conditions used. In all cases, duplicate experiments gave rate constants that varied by less that 50%.

The caged cAMP analogue, adenosine 3′,5′-cyclic monophosphate, P1-(2-nitrophenyl)ethyl ester (Calbiochem), was resuspended in dimethylsulfoxide (DMSO) to yield a 100× stock solution (200 mM). Dissolved analogue was delivered to the ribozyme reaction to yield final concentrations of 2 mM, and the resulting reaction mixture was supplemented with DMSO to give a final concentration of 5% to prevent its precipitation. This concentration of DMSO had no affect on the function of the clone cAMP-3. UV irradiation of the samples contained in a polycarbonate microtiter plate (USA Scientific) was conducted using a UV transilluminator (Spectroline model TVC-312A) that produces light centered at 312 nm. Under these conditions, greater than 80% of the analogue is converted to cAMP.

The cAMP depletion reactions were prepared by delivering cAMP (500 μM), 3′,5′-cyclic nucleotide phosphodiesterase (activator deficient from bovine brain, Sigma) and calmodulin (3′,5′-cyclic nucleotide phosphodiesterase activator, Sigma) as indicated for each reaction. Lyophilized phosphodiesterase and calmodulin samples were separately resuspended in a buffer containing 50 mM MES (pH 6.5 at 23° C.), 100 mM NaCl and 60% glycerol. Phosphodiesterase was delivered as indicated to a final concentration of 5×10⁻⁴ Uμl⁻¹ and calmodulin was delivered as indicated to a final concentration of 1.5 Uμl⁻¹. Reactions for the cAMP depletion studies contained 50 mM Tris-HCl (pH 7.5 at 23° C.), 20 mM MgCl₂, 30 μM CaCl₂, and 2.7% glycerol. Trace amount of internally ³²P labeled cAMP-1 RNA was added immediately (no preincubation) or was added after a 40 or 80 min preincubation that was carried out at 301C.

Results and Discussion

Beginning with a pool of 10¹⁵ RNA molecules representing nearly all possible sequence variants within the random-sequence domain of the construct, successive negative and positive selection reactions were conducted using a mixture of the four natural 3′,5′-cyclic mononucleotides (cNMPs; 500 μM each) as potential effector molecules. Each RNA population was prepared by in vitro transcription in the absence of the cNMP mixture and the full-length precursor RNAs were purified by denaturing 10% polyacrylamide gel electrophoresis (PAGE). The isolated RNA precursors were incubated in the absence of the effector mixture under otherwise permissive reaction conditions (reaction buffer: 50 mM Tris-HCl, pH 7.5 at 23° C., and 20 mM Mg²⁺) for an extended period of time. Uncleaved precursors from this negative selection reaction were again isolated by PAGE and subjected to positive selection by brief incubation under the permissive reaction conditions containing the cNMP mixture. The resulting 5′-cleavage products were purified by PAGE and amplified by reverse transcription followed by the polymerase chain reaction (RT-PCR). This selective-amplification process was repeated to favor the enrichment of allosteric ribozymes that respond to any of the four cNMPs.

Acid-Sensitive and Effector-Independent Ribozymes. After only six rounds of selective amplification (G6), the RNA pool exhibited a significant positive response to the addition of the cNMP mixture (FIG. 9b). However, upon further examination, it was found that the G6 RNA population does not specifically recognize any of the cNMPs, but is dominated by ribozymes that are triggered to function by a brief acidic treatment. Over the first six rounds of selection, the pH of the RNA mixture had been unintentionally lowered by adding an acidic mixture of cNMPs immediately prior to the addition of the reaction buffer. To prevent acidification, the RNA pool used for the positive selection was buffered with 50 mM Tris-HCl (pH 7.5 at 23° C.) prior to the addition of the cNMP mixture and the 20 mM Mg²⁺ used to initiate the reaction.

Two additional classes of selfish RNA molecules also became evident in the early stages of selection. One class of selfish ribozymes promote the RNA cleavage reaction with substantially reduced catalytic rates in both the negative and positive selection steps. The other class distributes into properly folded and misfolded states. In both cases, the ribozymes are not completely self-processed during the negative selection reaction, and therefore are enriched by the selective-amplification process without responding to the effectors. These two types of selfish RNAs contributed to the high background level of RNA catalysis that was observed in the positive selection reaction, and this rendered the efficiency of the allosteric selection process less than optimal.

Fortunately, ribozymes that specifically activate by recognizing an effector molecule attain a significant selective advantage over ribozymes that employ the effector-independent strategies described above. Extension of the incubation time for the negative selection reaction was used to further disfavor ribozymes that cleave more slowly. However, ribozymes that persist using a misfolding strategy were more difficult to eliminate. Presumably, a certain portions of these molecules partition into active and inactive conformational states after each denaturation event. Therefore, only part of the population cleaves during the negative selection. Upon purification of the uncleaved. precursors by denaturing PAGE, the RNAs have another chance to refold and distribute between the two conformational states. This allows a significant portion of the population to cleave during the subsequent positive selection reaction. To disfavor ribozymes that employ this strategy, multiple rounds of negative selection and purification were conducted. Alternatively, negative selection reactions were interspersed with thermal or chemical denaturation steps to cleave and refold the RNAs repetitively (see Materials and Methods above).

Isolation of cNMP-Dependent Hammerhead Ribozymes. A measurable response to the cNMP mixture was once again exhibited by the selected RNA populations after a total of 14 rounds (FIG. 9B). The G16 RNA pool was observed to be dominated by allosteric ribozymes that are activated specifically upon the addition of cGMP. Therefore, an additional two rounds of selection using only cGMP as the effector. The resulting population, termed G18′ RNA, is highly responsive to the addition of cGMP (FIG. 9C).

To recover ribozymes that respond to the remaining cNMPs, cGMP was added to the negative selection reaction at G17 and supplied the remaining three effectors in the positive selection reaction. By G19, the RNA pool no longer responds to cGMP, but shows specificity for cCMP. Therefore, an additional round of selection using only cCMP as the effector was conducted to produce G20′ RNA. This RNA population preferentially cleaves in the presence of cCMP (FIG. 9C).

In a repetition of this strategy, both cGMP and cCMP were included in the negative selection beginning with G20, while supplying cAMP and cUMP in the positive selection. This process yielded a population of RNAs at G22 that now responds positively to cAMP. An additional round of selection using only cAMP gave rise to G23′ RNA, a population that exhibits allosteric activation exclusively by this effector (FIG. 9C). However, after conducting an additional six rounds of selection using only cUMP in the positive selection reaction, specific enhancement in RNA cleavage by this effector was not observed. This finding indicates that cUMP-specific ribozymes were not present in the initial population and that ribozymes with this effector specificity did not by chance emerge as a result of mutations acquired during the selective-amplification process.

Kinetic Modulation of Ribozymes with cGMP, cCMP and cAMP.

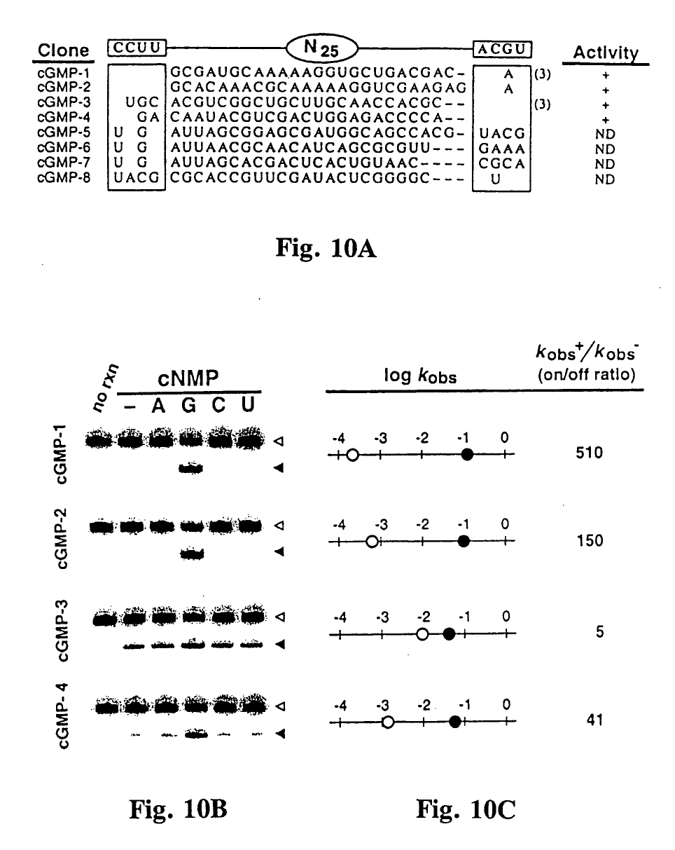

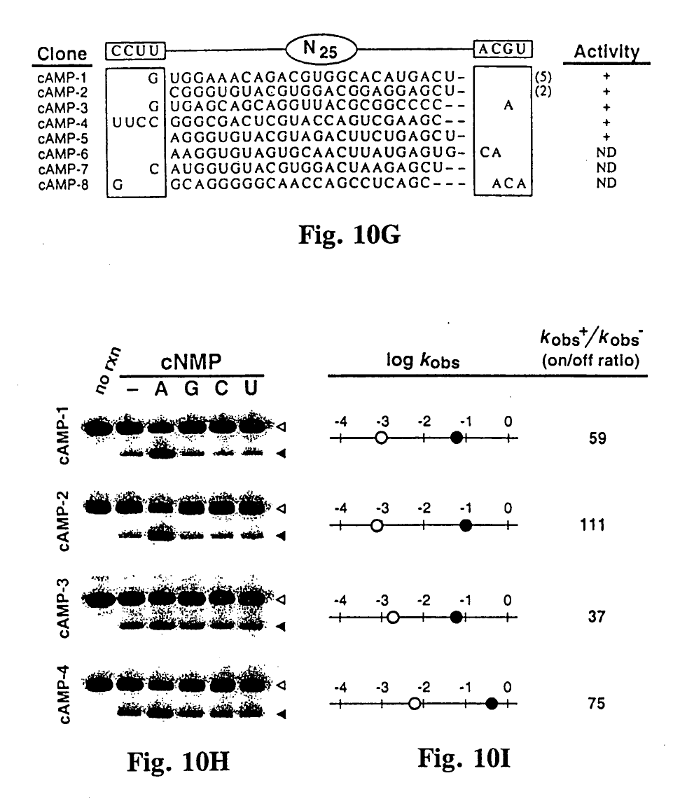

Clones from the G18′, G20′ and G23′ populations were sequenced in order to further characterize the function of the selected RNAs. Of the 12 clones examined from the G18 population, eight display considerable diversity within the original random-sequence domain (FIG. 10A). Interestingly, all individuals sustained at least one mutation within the regions that define the communication module, and all but one clone carry deletions within the random-sequence domain. This finding indicates that the original pool may not have offered a significant representation of allosteric ribozymes for the cNMP targets despite our efforts to bias the design of the RNA construct in favor of allosteric function.

Clones cGMP-1 through cGMP-4 were tested for catalytic activity and each responds positively to the addition of cGMP with distinctive characteristics (FIG. 10B). A comparison of the initial rates of hammerhead cleavage measured in the absence and the presence of effector (without regard for non-linear kinetics) reveal that cGMP-1 is activated ˜510 fold under the conditions used for allosteric selection (FIG. 10C). The remaining three clones are activated to a lesser magnitude, however each exhibits selective activation with cGMP and shows no cross reactivity with the remaining non-cognate effector molecules.

Similarly, individual clones from the G20′ and G23′ populations demonstrate specific activation with cCMP and cAMP effectors, respectively. As observed with the cGMP-specific RNAs, the sequences of the isolated G20′ RNAs reveal the acquisition of significant mutations or deletions over the course of the selection process, indicating that these changes may have been necessary to give rise to allosteric function (FIG. 10D). Although the catalytic performance of all seven clones sequenced from G20′ were examined, only cCMP-1 and cCMP-2 were observed to be activated by its corresponding effector (FIGS. 10E and 10F). The remaining clones manifest weak catalytic activity without regard to the presence of any effector, indicating that these RNAs have persisted to this stage in the selection process without utilizing an allosteric activation strategy.

Eight distinct individuals were also identified among the 13 clones sequenced from the G23′ population (FIG. 10G). Again, the clones have experienced significant acquisition of mutations within the original communication module or deletions within the random-sequence domains. Each of the five clones examined from the G23′ population respond positively to the presence of cAMP (FIG. 10H). Moreover, the clones cAMP-1 through cAMP-4 display allosteric, reaction kinetics that are similar to those observed with the previous allosteric constructs (FIG. 101). Although no cUMP-dependent ribozymes were isolated from this RNA population, the diversity of sequences and kinetic characteristics of the allosteric ribozymes that were recovered indicate that significant potential exists for the generation of novel effector-modulated RNAs.

Molecular Recognition by Effector Binding Sites. Of primary concern is whether the representative cGMP-, cCMP- and cAMP-dependent ribozymes directly recognize the atomic structures of their corresponding effectors, or whether they respond to some other physicochemical signaling agent that might be unintentionally introduced into the reaction mixture. Precedence for alternative effectors for allosteric activation is provided by the observation that the first ribozymes that dominated the RNA population do not respond specifically to any of the four cNMPs, but are sensitive to acidification of the reaction mixture. To determine if the mechanism of ribozyme activation is mediated through direct molecular recognition of cNMPs, adenosine 3′,5′-cyclic monophosphate, P1-(2-nitrophenyl)ethyl ester, a “caged” form of cAMP was used (FIG. 11A). The caged cAMP is a triester analogue of cAMP similar to those reported by Nerbonne, et al. (67) and is uncaged by cleavage of the added phosphoester linkage by irradiation with ultraviolet light. This caged effector provides a means to test whether an individual cAMP-dependent clone can be activated upon releasing the effector by irradiation.

The cAMP-dependent clones cAMP-1, cAMP-2 and cAMP-4 (FIG. 101) each cleave when presented with the caged effector (data not shown), suggesting that the allosteric binding sites of these RNAs accommodate the chemical alteration present in this analogue of cAMP. In contrast, the cAMP-3 clone exhibits the same rate constant whether it is incubated with 500 μM caged cAMP or whether it is incubated in the absence of effector (FIG. 11B). Presumably, the allosteric binding site of cAMP-3 excludes the caged cAMP compound from binding and activating the adjoining ribozyme. However, brief irradiation of a mixture containing cAMP-3 RNA and the caged cAMP with long wave UV light centered on ˜312 nm results in a significant activation of ribozyme function. The finding that UV-induced production of cAMP in situ triggers ribozyme activation is consistent with a mechanism whereby cAMP is directly recognized as an effector by this particular allosteric ribozyme.

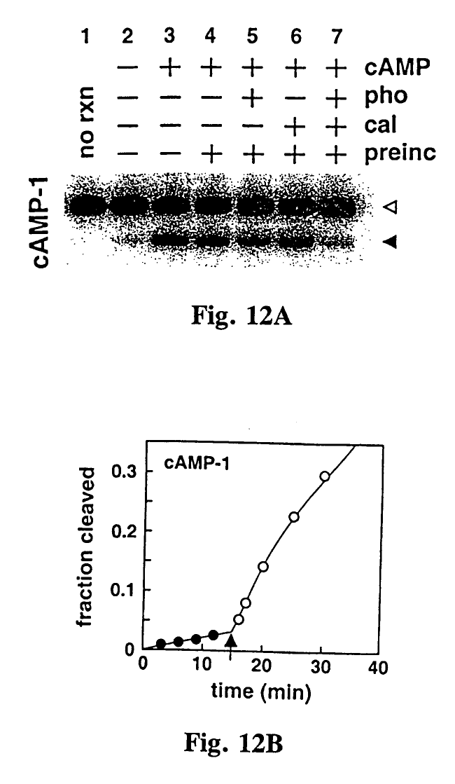

To further investigate whether molecular recognition of cNMP effectors by RNA mediates allosteric ribozyme function, an assay wherein cAMP is depleted from the reaction mixture in situ was established (FIG. 12). The in situ depletion of cAMP was achieved using cyclic nucleotide phosphodiesterase (68) and its activator calmodulin. These proteins do not deplete the effector when incubated independently, but when combined they efficiently hydrolyze 3′,5′-cyclic AMP to yield 5′-AMP. Under the assay conditions less than 10% of the cAMP is destroyed during a 40 min preincubation in the presence of the phosphodiesterase alone, however more than 90% is destroyed in a similar reaction containing calmodulin, an activator of cyclic nucleotide phosphodiesterase activity.

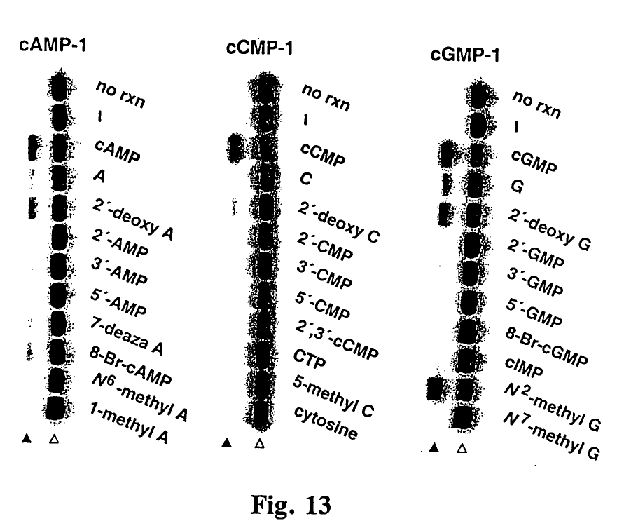

The allosteric ribozyme cAMP-1 does not accommodate 5′-AMP as an effector (see FIG. 13). As a result, this ribozyme should not be activated if cAMP is first depleted by the catalytic action of phosphodiesterase/calmodulin complexes. As expected, we find that neither phosphodiesterase nor calmodulin alone inhibit allosteric activation of cAMP-1 RNA (FIG. 12A, lanes 5 and 6). In contrast, the allosteric ribozyme is not significantly activated when added to a reaction mixture containing cAMP that has been preincubated with both phosphodiesterase and calmodulin (FIG. 12A, lane 7). Moreover, it was observed that cAMP-1 ribozymes in a reaction mixture equivalent to that used for lane 7 could be activated upon addition of a second aliquot of cAMP (FIG. 12B). This indicates that the loss of ribozyme activation upon preincubation with both protein factors is caused by the depletion of cAMP effector and is not due to any inhibitory effects that are inherent to the protein complex. Both studies described above, which involve either in situ production or depletion of cAMP, provide evidence that at least some of the many ribozymes isolated by allosteric selection directly recognize their corresponding cNMP effector molecules.

Molecular Discrimination by Allosteric Binding Sites. A preliminary survey of the molecular recognition determinants was conducted using representative clones cGMP-1, cCMP-1 and cAMP-1. In each case, the RNAs exhibit significant discrimination against closely related analogues of their corresponding effector (FIG. 13). For example, cGMP-1 RNA shows significant discrimination against 3′-GMP and 5′-GMP, the hydrolyzed analogues of cGMP. Likewise, the cCMP-1 and cAMP-1 clones also exhibit this same ability to distinguish whether the cyclic phosphodiester structure of their corresponding cNMP effectors has been opened by hydrolysis of the 5′ O—P or the 3′ O—P bonds.

Although additional experimentation is necessary to more clearly define the determinants of molecular recognition for these allosteric ribozymes, it appears that in each case the discrimination against opened-ring analogues could be due to steric interactions. The observation that all three clones remain at least partially active when supplied with the corresponding nucleoside and deoxynucleoside analogues of cNMP indicates that the phosphate moiety is not absolutely required for allosteric activation. In contrast, alteration of many of the functional groups on the nucleotide base of each effector adversely affects allosteric ribozyme function (FIG. 13). Therefore, the base moieties of the cNMP effectors appear to be essential for molecular recognition by the different effector-binding domains.

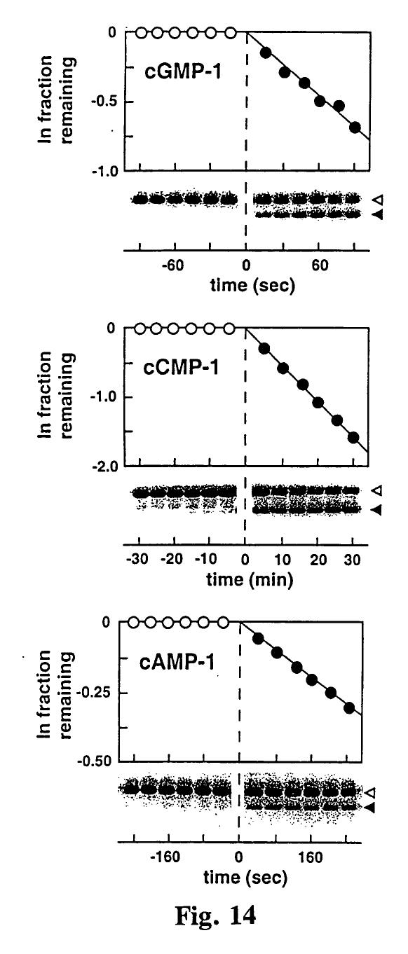

Rapid Activation of cNMP-Dependent Ribozymes. A common characteristic of the small-molecule-dependent allosteric ribozymes created to date is the rapid activation or deactivation of ribozyme function upon addition of the effector (5, 7, 65). The rapid allosteric response is a kinetic feature that is highly desirable for RNA molecular switches that are to find practical application. Therefore, the activation kinetics for the three representative clones cGMP-1, cCMP-1 and cAMP-1 were examined. In each case, the ribozymes appear to be activated within seconds after introduction of their corresponding effector molecules (FIG. 14). Rapid activation of ribozyme function is indicative of a dynamic RNA structure that quickly forms active effector-binding and ribozyme conformations only upon introduction of the appropriate signaling agent.

Each of the clones described above maintain linear cleavage kinetics through at least one half life (FIG. 14), indicating that greater than 50% of an individual clone's RNAs are activated upon addition of the appropriate effector. However, self-cleavage for some individuals reaches a plateau after only a short reaction time, which might be indicative of significant misfolding problems. Upon allosteric activation, most clones examined undergo between 20% to 90% processing before cessation of catalysis.

Binding Affinities and Dynamic ranges. The effector-binding site of each allosteric ribozyme is expected to bind its ligand with a distinct affinity that can be described by a dissociation constant (KD) for the RNA-ligand interaction. If occupation of the effector-binding site indeed correlates with the level of activation for a particular allosteric ribozyme, then an apparent KD for effector binding can be established for this interaction by examining the dependency of catalytic rate on the concentration of effector.

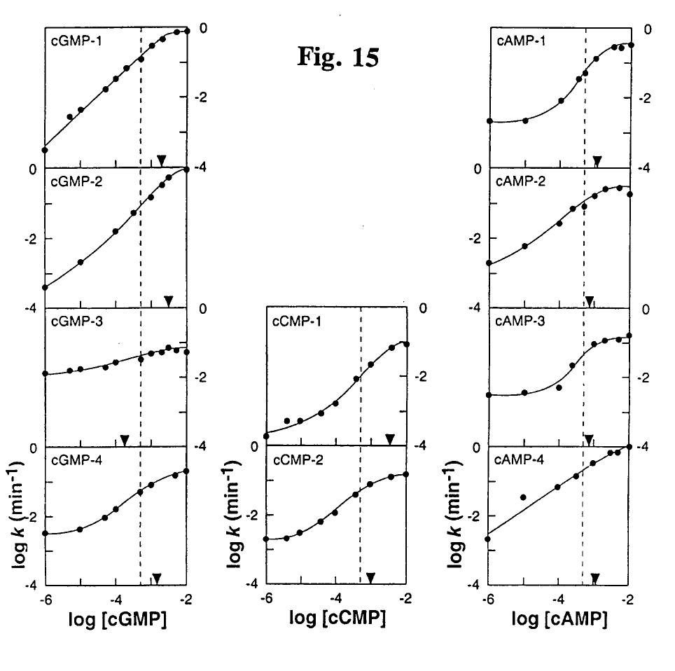

To provide a comprehensive analysis of the binding affinities displayed by the allosteric ribozymes that were isolated in this study, the effector concentration-dependent activities of all ten allosteric ribozymes described in FIGS. 11 to 13 were determined. Apparent KD values were determined by establishing the effector concentration that produces a rate constant that is half maximal (½ kₘₐₓ). In all cases, the apparent KD falls near the concentration of each effector used during in vitro selection (FIG. 15). These constants range from ˜200 μM (cGMP-3) to ˜4 mM (cCMP-1). By comparison, most ligand-binding RNAs isolated by SELEX methods (16, 46-53) bind with higher affinities, indicating that improvements in the sensitivity of these allosteric ribozymes to lower concentrations of effector could be achieved.

The plots used to define the apparent KD for each allosteric ribozyme (FIG. 11) also reveal the range of rate constants that are exhibited for different concentrations of effector. This “dynamic range” for allosteric responses is highly variable between the different clones, suggesting that the diversity of functional characteristics that can be manifested by allosteric ribozymes is substantial. As expected from the preliminary analysis (FIG. 10B), the cGMP-3 ribozyme has a poor rate enhancement or “allosteric response” to cGMP. As a result, this individual exhibits an overall dynamic range of less than one order of magnitude. In contrast, the clone that displays the best dynamic range is cGMP-1, which maintains a linear increase in the logarithm of its rate constant from 1 μM through 1 mM. Although the increase in the rate constant for cGMP-1 under in vitro selection conditions is ˜500 fold, the overall rate increase upon saturation of the effector-binding site with cGMP is approximately 5,000 fold. This corresponds to a dynamic range for cGMP-1 of greater than three orders of magnitude.

Engineering Novel RNA Molecular Sensors. The allosteric selection strategy (FIG. 9A) employed in this study provides an alternative approach for the isolation of novel multidomain RNAs that function as molecular switches, and for the isolation of new ligand-binding RNA structures. The simultaneous isolation of numerous allosteric ribozymes that respond to particular cNMP targets are reported herein. Similarly, allosteric selection could be used for the isolation of molecular sensors on a massively parallel scale by using mixtures of metal ions and metal complexes or by using complex mixtures containing hundreds of organic compounds, proteins or nucleic acids as candidate effector molecules in the positive selection reaction. Indeed, any physicochemical impulse that can influence RNA structure folding could be a signalling agent for allosteric ribozyme function.

Structural and Functional Versatility of RNAs. In contrast to the limited functions of natural ribozymes, protein enzymes catalyze a tremendous array of chemical transformations with extraordinary precision and enormous rate enhancements. Included among the diverse biochemical functions of protein enzymes are conformational. changes that in some instances provide effector-dependent allosteric modulation (21). Unlike their protein counterparts, natural ribozymes are not known to undergo allosteric modulation of catalytic activity. However, the results of this study and several earlier studies (5, 6, 8, 9, 61-63, 65, 66) provide evidence that nucleic acids are quite capable of modulating catalytic activity in response to various effector compounds. These findings are consistent with earlier suggestions (57-60) that RNA may have significant untapped potential for complex catalytic function. Presumably, the true catalytic potential of nucleic acids can be harnessed for the construction of synthetic ribozymes that make unique biochemical applications possible.

It is important to note that the allosteric ribozymes described in this study have not been subjected to any efforts to optimize their allosteric responses and catalytic function. Illustrated are representative clones that were generated by this initial in vitro selection process, regardless of their kinetic characteristics, in order to give a sense of the properties of allosteric ribozymes that first proved successful. The ribozymes described in this example should be considered prototypic because in most cases their effector binding affinities and catalytic rates are most likely inadequate to serve in most applications. Presumably, individual classes of allosteric ribozymes isolated by allosteric selection will be amenable to further optimization using similar in vitro selection strategies like those used in this study. This would ultimately allow their development as efficient molecular sensors for various applications.

Implications for the Control of Gene Expression. Precise control over gene expression is of profound importance to the normal function of all cells.

Likewise, the purposeful manipulation of gene expression that is directed with precise temporal or spatial command is of great interest to those who desire to control biological systems at the molecular level. Conceivably, the regulation of gene expression can occur at any stage of the process of information transfer from DNA to RNA and from RNA to the final protein product. In fact, natural systems have evolved an abundance of strategies that are used to adjust the levels of gene accessibility and to modulate the molecular processes that occur after transcription (69). Many of these mechanisms have become targets for the development of small-molecule regulators that can be used to control gene expression (70).

A number of genetic control mechanisms of cells are exerted at the level of RNA. Natural antisense interactions and the modulation of RNA stability, for example, are two mechanisms that are known to impact gene expression. Antisense oligonucleotides and ribozymes are widely used by investigators to purposefully influence the expression of specific genes by exploiting these two mechanisms. These approaches modulate RNA function either by sterically blocking access to the RNA target or by targeting the RNA for destruction. Recently, it was shown that mRNA translation could be blocked by exploiting specific interactions between aptamers and certain dye compounds (71). Specifically, RNA aptamers that selectively bind Hoechst dyes H33258 and H33342 were integrated into mRNAs such that gene expression was selectively blocked when these ligands were introduced to the cell. Similarly, allosteric ribozymes could be fused to mRNAs so that when the corresponding effector molecule is introduced into the cell, the ribozyme domain adjusts its catalytic activity. Therefore, allosteric effector molecules could be used to modulate the stability of mRNAs and thus influence the expression of a target gene.

The allosteric selection protocol described herein makes possible the simultaneous selection of new allosteric ribozymes that respond to any of hundreds or even thousands of compounds. This provides a means to test whether self-cleaving ribozymes such as the hammerhead can be made to respond to a wide range of effector stimuli and whether the resulting allosteric constructs can be integrated with mRNAs as new genetic control elements. If this proves feasible, then nearly any natural or bioavailable compound is a candidate for the purposeful control of gene expression in genetically transformed organisms.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

### REFERENCES

1. McDaniel, R., et al. (1995) Nature 375, 549-554.
2. Marsden, A. F. A., et al. (1998) Science 279, 199-202.
3. Hellinga, H. W. & Marvin, J. S. (1998) Trends Biotechnol. 16, 183-189.
4. Crane, D. E., et al. (1998) Science 282, 63-68.
5. Tang, J. & Breaker, R. R. (1997) Chem. Biol. 4, 453-459.
6. Tang, J. & Breaker, R. R. (1997) RNA 3, 914-925.
7. Araki, M., et al. (1998) Nucleic Acids Res. 26, 3379-3384.
8. Tang, J. & Breaker, R. R. (1998) Nucleic Acids Res. 26, 4214-4221.
9. Soukup, G. A. & Breaker, R. R. (in press, 1999) Allosteric ribozymes. In Ribozymes: Biology and Biotechnology. (eds. Gaur, R. K. & Krupp, Eaton Publishing.)
10. Gerstein, M., et al. (1994) Biochemistry 33, 6739-6749.
11. Pyle, A. M. & Green, J. B. (1995) Curr. Biol. 5, 303-310.
12. Draper, D. E. (1996) Trends Biochem. Sci. 21, 145-149
13. Westhof, E., et al. (1996) Fold. Des. 1, 78-88.
14. Williams, K. P. & Bartel, D. P. (1996) Nucleic Acids Mol. Biol. 10, 367-381.
15. Breaker, R. R. (1997) Chem. Rev. 97, 371-390.
16. Gold, L., et al. (1995) Annu. Rev. Biochem. 64, 763-797.
17. Osborne, S. E. & Ellington, A. D. (1997) Chem. Rev. 97, 349-370.
18. Monod, J. & Jacob, F. (1961) Cold Spring Harbor Symp. Quant. Biol. 26, 389-401.
19. Monod, J., Changeux, J.-P. & Jacob, F. (1963) J. Mol. Biol. 6, 306-329.
20. Kurganov, B. I. (1978) Allosteric Enzymes. John Wiley & Sons Ltd., New York, N.Y.
21. Perutz, M. (1994) Mechanisms of Cooperativity and Allosteric Regulation in Proteins. (Cambridge University Press, New York, N.Y.)
22. Ye, X., et al. (1996) Nature Struct. Biol. 3, 1026-1033.
23. Hsiung, C. & Patel, D. J. (1996) Nature Struct. Biol. 3, 1046-1050.
24. Lin, C. H. & Patel, D. J. (1997) Chem. Biol. 4, 817-832.
25. Patel, D. J., et al. (1997) J. Mol. Biol. 272, 645-664.
26. Burgstaller, P. & Famulok, M. (1994) Angew. Chem. Int. Ed. Engl. 33, 1084-1087.
27. Forster, A. C. & Symons, R. H. (1987) Cell 49, 211-220.
28. Fedor, M. J. & Uhlenbeck, O. C. (1992) Biochemistry 31, 12042-12054.
29. Tuschl, T. & Eckstein, F. (1993) Proc. Natl. Acad. Sci. USA 90, 6991-6994.
30. Long, D. M. & Uhlenbeck, O. C. (1994) Proc. Natl. Acad. Sci. USA 91, 6977-6981.
31. Burgstaller, P., et al. (1997) Nucleic Acids Res. 25, 4018-4027.
32. Carey, J. (1988) Proc. Natl. Acad. Sci. USA 85, 975-979.
33. Burgstaller, P. & Famulok, M. (1996) Bioorg. Med. Chem. Lett. 6, 1157-1162.
34. Fan, P., et al. (1996) J. Mol. Biol. 258, 480-500.
35. Serra, M. J. & Turner, D. H. Meth. Enzymol. 259, 242-261.
36. Lodmell J. S. & Dahlberg, A. E. (1997) Science 277, 1262-1267.
37. von Ahsen, U. (1998) Chem. Biol. 5, R3-R6.
38. Jenison, R. D., et al. (1994) Science 263, 1425-1429.
39. Sassanfar, M. & Szostak, J. W. (1993) Nature 364, 550-553.
40. Famulok, M. (1994) J. Am. Chem. Soc. 116, 1698-1706.
41. Breaker, R. R. & Joyce, G. F. (1994) Trends Biotechnol. 12, 268-275.
42. Ruffner, D. E., et al. (1990) Biochemistry 29, 10695-10702.
43. U.S. Pat. No. 5,589,333 to A. Shih, et al. (1996).
44. WO 98/08974 by N. Asher, et al. (Intelligene, 1998).
45. WO 98/27104 by R. Breaker (Yale University, 1998).
46. U.S. Pat. No. 5,567,588 to L. Gold & S. Ringquist (1996).
47. U.S. Pat. No. 5,637,459 to D. Burke, et al. (1997).
48. U.S. Pat. No. 5,712,375 to K. B. Jensen, et al. (1998).
49. U.S. Pat. No. 5,723,289 to B. E. Eaton & L. Gold (1998).
50. U.S. Pat. No. 5,723,592 to B. E. Eaton & L. Gold (1998).
51. U.S. Pat. No. 5,763,177 to L. Gold, et al. (1998).
52. U.S. Pat. No. 5,763,566 to K. B. Jensen, et al. (1998).
53. U.S. Pat. No. 5,773,598 to D. Burke, et al. (1998).
54. Pan, T. (1997) Curr. Opin. Chem. Biol. 1, 17-25.
55. Frauendorf, C. & Jaschke, A. (1998) Angew. Chem. Int. Ed. 37, 1378-1381.
56. Kurz, M. & Breaker, R. R. (1999) Current Topics in Microbiology and Immunology, Vol. 243: Combinatorial Chemistry in Biology, (eds. Winnacker, E.-L., et al., Springer-Verlag, Berlin, Germany) 137-156.
57. White, H. B. 111 (1976) J. Mol. Evol. 7, 101-104.
58. Benner, S. A., et al. (1987) Cold Spring Harbor Symp. Quant. Biol. 52, 53-63.
59. Benner, S. A., et al. (1989) Proc. Natl. Acad. Sci. USA 86, 7054-7058.
60. Hirao, I. & Ellington, A. D. (1995) Curr. Biol. 5, 1017-1022.
61. Porta, H., & Lizardi, P. M. (1995) Bio/Technol. 13, 161-164.
62. Kuwabara, T., et al. (1998) Molecular Cell 2, 617-627.
63. Robertson, M. P., & Ellington, A. D. (1999) Nature Biotechnol. 17, 62-66.
64. Batey, R. T. & Doudna, J. A. (1998) 5, 337-340.
65. Soukup, G. A. & Breaker, R. R. (1999) Proc. Natl. Acad. Sci. USA 96, 3584-3589.
66. Soukup, G. A. & Breaker, R. R. (1999) Structure Fold. Des. 7, 783-791.
67. Nerbonne, J. M., et al. (1984) Nature 310, 74-76.
68. Ho, H. C., et al. (1976) Biochim. Biophys. Acta 429, 461-473.
69. Lin, E. C. C. & Lynch, A. S. (1996) Regulation of gene expression in Escherichia coli. (R. G. Landes Co, Austin, Tex.).
70. Denison, C. & Kodadek, T. (1998) Chem. Biol. 5, R129-R145.
71. Werstuck, G. & Green, M. R. (1998) Science 282, 296-298.
72. Hertel, K. J., et al. (1992) Nucleic Acids Res. 20, 3252.
73. Koizumi, M., et al., (1999) Nat. Struc. Bio. 11, 1-10

The papers and patents cited herein are expressly incorporated in their entireties by reference.

1. A purified functional polynucleotide comprising an actuator domain, a receptor domain, and a bridging domain, wherein interaction of the receptor domain with a signalling agent triggers a conformational change in the bridging domain which modulates the activity of the actuator domain.

2. A polynucleotide according to claim 1 wherein the signalling agent is a ligand that binds to the receptor domain.

3. A polynucleotide according to claim 1 wherein the activity of the actuator domain is catalytic.

4. A polynucleotide according to claim 1 wherein at least two of the domains are non-overlapping.

5. A polynucleotide according to claim 1 wherein at least two of the domains are partially or completely overlapping.

6. A polynucleotide according to claim 1 which is RNA.

7. A polynucleotide according to claim 6 which is a hammerhead ribozyme.

8. A polynucleotide according to claim 1 which is DNA.

9. A polynucleotide according to claim 1 wherein the actuator domain exhibits catalytic activity that is triggered by binding of a chemical compound to the receptor domain.

10. A biosensor comprising a polynucleotide according to claims 1, 2, 3, 4, 5, 6, 7, 8, or 9.

11. A biosensor according to claim 10 in which the polynucleotide is attached to a solid support.

12. A method for detecting the presence or absence of a ligand or its concentration in a sample comprising contacting the sample with a polynucleotide according to claims 1, 2, 3, 4, 5, 6, 7, 8, or 9.

13. A method according to claim 12 wherein the presence or absence of a ligand or its concentration is determined by observation of a chemical reaction.

14. A method according to claim 12 wherein the presence or absence of a ligand or its concentration is detected by observation of a change in polynucleotide configuration or function.

15. A process for preparing polynucleotides that are responsive to the presence or absence of a signalling agent, comprising linking a polynucleotide actuator domain, a receptor domain, and a bridging domain together such that interaction of the signalling agent with the receptor domain triggers a conformational change in the bridging domain which modulates the activity of the actuator domain.

16. A process according to claim 15 wherein the receptor domain has a ligand binding site and wherein ligand binding triggers a confirmational change in the bridging domain that stimulates catalytic activity of the actuator domain.

17. A process for screening polynucleotides which have an actuator domain, a receptor domain, and a bridging domain and which are responsive to a signalling agent in a sample, comprising linking a bridging domain having defined properties that modulate the activity of a corresponding actuator domain having defined properties, to a receptor domain having a random sequence, and identifying polynucleotides responsive to the signalling agent by incubation of the sample with the polynucleotide so constructed by observation of modulation of the activity of the actuator domain.

18. A process according to claim 17 wherein the receptor domain has a ligand binding site and wherein ligand binding triggers a confirmational change in the bridging domain that stimulates catalytic activity of the actuator domain.

19. A process for preparing RNA sensors according to any of claims 15, 16, 17, or 18.

20. A process for preparing DNA sensors according to any of claims 15, 16, 17, or 18.
